(19) United States
(12) Patent Application Publication
(10) Pub. No.: US 2020/0370095 A1
(43) Pub. Date: Nov. 26, 2020

(54) Spatial Analysis

(71) Applicant: Takara Bio USA, Inc., Mountain View, CA (US)

(72) Inventors: Andrew Alan Farmer, Los Altos, CA (US); George G. Jokhadze, Mountain View, CA (US); Alain Mir, Mountain View, CA (US)

(21) Appl. No.: 16/882,357

(22) Filed: May 22, 2020

Related U.S. Application Data
(60) Provisional application No. 62/852,741, filed on May 24, 2019.

(51) Int. Cl. C12Q 1/6806; C12Q 1/6876

(52) U.S. Cl. CPC C12Q 1/6806; C12Q 2600/156; C12Q 1/6876

(57) ABSTRACT

The invention pertains to methods for assessing a section of a biological sample, e.g., for determining the location of an analyte in the section. In certain embodiments, the methods comprise: a) contacting the biological sample section with an oligonucleotide indexed surface comprising an addressable array of capture oligonucleotides; b) probing the oligonucleotide indexed surface contacted with the biological sample section with an analyte-specific binding member that specifically binds to the analyte, wherein the analyte-specific binding member comprises a detector oligonucleotide; c)linking the detector oligonucleotide to a barcoded capture oligonucleotide proximal thereto to produce a linked product nucleic acid, e.g., a ligated nucleic acid or an extension product nucleic acid; and d) sequencing the linked product nucleic acid to assess the biological sample for the analyte. Kits for carrying out the methods of the invention are also provided. Further provided are systems configured for carrying out the methods disclosed herein.

### CROSS-REFERENCE

Pursuant to 35 U.S.C. § 119(e), this application claims priority to the filing date of U.S. Provisional Patent Application Ser. No. 62/852,741, filed May 24, 2019; the disclosure of which application is herein incorporated by reference.

### INTRODUCTION

Several methods are known for obtaining single cell genomics data from individual cells. Parallel sample preparative methodologies reducing batch effects have been developed. For example, single cell qPCR, massively multiplex targeted primer enriching methods, Takara Bio USA's SMARTer technology enabling examination of single cell, 3′ and 5′ end sequences, T-Cell receptor-based and, potentially, whole transcriptome-based RNA Seq have been developed. Examples of automated systems include Takara Bio USA's ICELL8/Cx systems, 10x Chromium, Drop-Seq, In-Drop technology and the Fluidigm C1 systems.

It is also possible to obtain single cell DNA profiles permitting both DNA and RNA copy number and SNV analyses. In the former case, Takara Bio USA's PicoPLEX technology is recognized as a gold standard and again there are applications from other sources. Other methods include Repli-g from Qiagen that uses Multiple Displacement Amplification (MDA) and Multiple Annealing and Looping Based Amplification Cycles approach (MALBAC) from Yikon Genomics. Methods for chromatin analysis include ATAC-Seq and Chip-Seq methods, such as Cut&Tag, allow for analysis of chromatin states and transcription factor binding at single cell levels. There are also methods for methylation analysis—such as post-bisulfite adapter tagging (PBAT). Protein detection in single cells at a genomics scale has also been enabled with methods such as Fluidigm's CyTOF technology or single cell western blotting developed by Amy E. Herr's group at U.C. Berkeley and now commercialized as ProteinSimple. Briefly, these methods employ dissociated cells and thus lose the original spatial information. There have been some attempts to address this issue most notably, the method developed by Spatial Transcriptomics (now part of 10×). Also, on the protein side, Fluidigm has developed a variant of CyTOF for spatial imaging of up to 37 proteins using their Hyperion system for Imaging Mass Cytometry™ (IMC™). Another alternative for Spatial RNA-Seq from the group of Dr. Kambara at Waseda University in Japan (see world-wide-website: //doi.org/10.1038/s41598-017-04616-6) uses a punch mechanism to isolate an array of FFPE tissue fragments for downstream RNA-Seq analysis. Additional methods include smFISH technologies such as MERFISH from Xiaowei Zhuang and seqFISH from Long Cai's group at Cal Tech. Other methodologies have been employed including Slide-Seq (Macosko group, Broad Inst), ReadCor technology and Spatially-resolved Transcript Amplicon Readout Mapping (STARmap) at Stanford University, while single cell mass spectroscopy has received National Institutes of Health funding. Nanostring's GeoMx™ Digital Spatial Profiling technology is another example that is arguably the most commercially advanced of these approaches.

However, regardless of the approaches described, these methods do not typically permit simultaneous examination of RNA and protein. Moreover, they are frequently limited in the number of analytes capable of being examined—e.g. a few 10ss to 100's by smFISH or up to 37 for Hyperion. These methods can also be expensive, mechanistically complex to perform, labor intensive or difficult to scale by automation.

Human bodies are composed of tens of trillions of specialized cells, structured into tissues. The organization, specialization, and cooperation of different cells within normal or diseased tissue—their spatial arrangement—has a profound impact on human health.

### SUMMARY

This disclosure provides methods for obtaining spatial arrangement “spatial profiling” data from multiple single cells, where a cell's location relative to other cells, that is, spatial context, or simply “spatial” location is important to know. The present disclosure provides methods for assessing a section of a biological sample (biological sample section), particularly, for determining the location of an analyte in the biological sample section.

In an aspect, the present disclosure provides a method for assessing a biological sample section comprising: a) contacting the biological sample section with an oligonucleotide indexed surface comprising an addressable array of capture oligonucleotides; b) probing the oligonucleotide indexed surface contacted with the biological sample section with an analyte-specific binding member that specifically binds to the analyte, wherein the analyte-specific binding member comprises a detector oligonucleotide; c) linking the detector oligonucleotide to a barcoded capture oligonucleotide proximal thereto to produce a linked product nucleic acid, which linked product nucleic acid may be: i) a ligated product nucleic acid made up ligated detector and barcode capture oligonucleotides, or ii) an extension product nucleic acid produced by template mediated extension of hybridized detector and barcode capture oligonucleotides; and d) sequencing the linked product nucleic acid to assess the biological sample for the analyte.

The biological sample section can be a paraffin embedded section or a frozen section. The biological sample section can also be fixed in a fixative, e.g., formalin, paraformaldehyde, glutaraldehyde, methanol, acetone, other appropriate fixative, e.g., as known in the art.

In one embodiment, each capture oligonucleotide comprises a barcode unique to the location of the capture oligonucleotide. The capture oligonucleotide can further comprise one or more additional functional domains, such as but not limited to (and in any convenient arrangement): a unique molecular barcode/index (UMI), a capture-detector hybridizing region, a capture-primer hybridizing region, a capture-splint hybridizing region, a cleavable linker, and a detectable label, a sequencing platform adapter construct, etc.

The oligonucleotide indexed surface can comprise a solid support of a material, such as glass, nitrocellulose, silicon, plastic, and a combination thereof.

The analyte assessed according to the method disclosed herein can vary, and in some instances may be a protein, nucleic acid, e.g., DNA, RNA, lipid, or carbohydrate. The analyte-specific binding member may also vary, with examples of such binding members being proteins, such as an antibody, nucleic acids, e.g., oligonucleotide, aptamer, polypeptides, carbohydrates, lipids, or small molecules.

In certain embodiments, the detector oligonucleotide comprises a barcode unique to the analyte. The detector oligonucleotide can further comprise one or more additional functional domains, such as but not limited to (and in any convenient arrangement): a detector-capture hybridizing region, a detector-primer hybridizing region, a unique molecular barcode/index (UMI), a detector-splint hybridizing region, a cleavable linker, a detectable label, and a sequencing platform adapter construct, etc.

Linking the detector oligonucleotide to the capture oligonucleotide proximal thereto can be performed using any convenient protocol.

Linking a capture oligonucleotide to a proximal detector oligonucleotide can be achieved by hybridization between the capture oligonucleotide and the detector nucleotide via regions in these oligonucleotides that hybridize with each other. Particularly, the free end of the capture oligonucleotide can have a sequence that hybridizes with the sequence at the free end of the detector oligonucleotide, thereby linking the capture oligonucleotide with the proximal detector oligonucleotide.

Linking a capture oligonucleotide to a proximal detector oligonucleotide can also be achieved via ligation. Ligation may be achieved using any convenient protocol and with any convenient ligase. Of interest are both splint-mediated and not splint mediated ligation protocols. In some instances, ligation can be achieved via a splint mediated protocol, e.g., a protocol which employs a splint oligonucleotide that hybridizes with both the capture oligonucleotide and the proximal located detector oligonucleotide. In some instances, the protocol includes hybridizing a splint oligonucleotide to: 1) a capture-splint hybridizing region on the capture oligonucleotide via a capture hybridizing region on the splint oligonucleotide that is complementary to capture-splint hybridizing region and 2) a detector-splint hybridizing region on the detector oligonucleotide via a detector hybridizing region on the splint oligonucleotide that is complementary to the detector-splint hybridizing region. A ligase can be used to ligate the capture-splint hybridizing region of the capture oligonucleotide and the detector-splint hybridizing region of the detector oligonucleotide. Ligating a detector oligonucleotide to a capture oligonucleotide produces a ligated product nucleic acid.

A linked product nucleic acid, or a template mediated polymerized derivative, e.g., amplicon, thereof, can be sequenced using any convenient protocol. In some instances, sequencing includes amplifying the linked product nucleic acid followed by sequencing of the resultant amplicons, e.g., by using a next generation sequencing method, such as paired-end sequencing, ion-proton sequencing, pyrosequencing, and nanopore sequencing.

In certain embodiments, one or more additional analytes can be assessed by using one or more additional analyte-specific binding members, wherein each additional analyte-specific binding member specifically binds to an additional analyte, and wherein each of the one or more additional analyte-specific binding members comprises an additional detector oligonucleotide, each additional detector oligonucleotide comprising a barcode unique to the additional analyte.

Assessing a biological sample section can comprise determining the location of the analyte and/or the one or more additional analytes in the biological sample section based on the presence in the linked product nucleic acid and/or the one or more additional linked product nucleic acids of the barcodes unique to the capture oligonucleotides and the barcodes unique to the detector oligonucleotide and/or the one or more additional detector oligonucleotides.

The location of the analyte and/or the one or more additional analytes in a plurality of biological sample sections can be used to determine the location of the analyte and/or the one or more additional analytes in a three-dimensional biological sample. The location of the analytes could be used to evaluate histology, pathology, or morphology of the biological sample.

Further embodiments of the invention provide a kit comprising at least one of: a) an oligonucleotide indexed surface comprising an addressable array of capture oligonucleotides; and b) an analyte-specific binding member that specifically binds to the analyte, wherein the analyte-specific binding member comprises a detector oligonucleotide.

Further embodiments of the invention provide a system comprising, a processing module configured to receive the following data: i) an image of a biological sample section, ii) sequences of capture oligonucleotides in an addressable array of capture oligonucleotides, wherein each capture oligonucleotide comprises a barcode unique to the location of the capture oligonucleotide on the array, and iii) sequences of linked product nucleic acids obtained by processing the biological sample section according to methods disclosed herein, wherein the processing module is configured to process the received data to determine the location of the analyte in the biological sample section. Processing modules may also be configured to receive information on the barcodes of the detectors used, e.g., as a table, for associating them to the analytes examined, so that the system can assign the analytes to the locations based on the barcodes of the detector and the capture oligo found in the product nucleic acids.

The system can be configured to receive data about a plurality of images for a plurality of biological sample sections to determine a location of one or more analytes in a three-dimensional biological sample.

### BRIEF DESCRIPTION OF THE FIGURES

FIG. 1 provides a schematic representation of an oligonucleotide indexed surface comprising an addressable array of capture oligonucleotides. The exemplary capture oligonucleotide comprises a disulfide link to allow conjugation of the oligonucleotide to the slide as well as separation from the slide, a capture-primer hybridizing region, a barcode unique to the location on the array of the capture oligonucleotide, and capture-splint hybridizing region. It is noted that any convenient method for putting oligos on a surface can be applied as appropriate.

FIG. 2 provides a schematic of certain steps of the methods disclosed herein. A FFPE sample is placed onto a slide having an oligonucleotide indexed surface comprising an addressable array of capture oligonucleotides. The FFPE sample is then probed with an antibody specific for an analyte, the antibody being conjugated to a detector oligonucleotide.

FIG. 3 provides a schematic of certain steps of an embodiment disclosed herein. A detector oligonucleotide conjugated to an antibody is brought into proximity with a capture oligonucleotide on the indexed surface. Particularly, the capture-splint hybridizing region and the detector-splint hybridizing region are proximate to each other.

FIG. 4 provides a schematic of certain steps of the methods disclosed herein. A splint oligonucleotide hybridizes with the capture oligonucleotide via capture-splint hybridizing region and to the detector oligonucleotide via the detector-splint hybridizing region. A ligase then ligates the end of the capture oligonucleotide to the end of the detector oligonucleotide to produce a ligated product nucleic acid.

FIG. 5 provides a schematic of certain steps of the methods disclosed herein. A ligated product nucleic acid can be amplified using a primer pair of a capture primer and a detector primer. Capture-primer hybridizes to the capture-primer hybridizing region of the capture oligonucleotide and a detector-primer hybridizes to the detector-primer hybridizing region of the detector oligonucleotide. Polymerase chain reaction produces copies of the ligated product nucleic acid. The amplified product contains a barcode from the capture oligonucleotide, which provides the information about the location, and a barcode from the detector oligonucleotide, which provides the information about the analyte. Based on the presence in an amplified product of a barcode sequence specific to a location and a barcode sequence specific to an analyte, one can determine the presence of the analyte at the position of the capture oligonucleotide.

FIG. 6 provides an alternative method where a second oligonucleotide indexed surface is laid onto a biological sample section laid onto an oligonucleotide indexed surface. The second oligonucleotide indexed surface comprises a second addressable array of capture oligonucleotides.

FIG. 7 provides an example of chemically cleavable capture oligonucleotide that can be released using reduction of a disulfide linker from the array surface

FIG. 8 shows fluorescence signal from AcGFP bound to a streptavidin-coated plate.

FIG. 9 shows Western Blot of JL-8 antibody conjugated to the biotinylated analyte-specific barcoded oligonucleotide (Lane 1) and JL-8 conjugated to non-biotinylated analyte-specific barcoded oligonucleotide, i.e., SEQ ID: 1 (Lane 2).

FIG. 10A shows chromatogram of the sequence generated from a ligated product nucleic acid using the forward primer.

FIG. 10B shows chromatogram of the sequence generated from a ligated product nucleic acid using the reverse primer.

FIG. 11 shows a schematic representation of certain embodiments of linking a capture oligonucleotide to a detector oligonucleotide via regions in these oligonucleotides that hybridize with each other.

FIG. 12 shows a schematic representation of certain embodiments of synthesizing an extension product nucleic acid, i.e., nucleic acid produced by template mediated extension of hybridized detector and barcode capture oligonucleotides, comprising the sequences of a capture oligonucleotide and a proximally located detector oligonucleotide. In this embodiment, the template mediated polymerized derivative, or an amplicon thereof, may be sequenced, as desired.

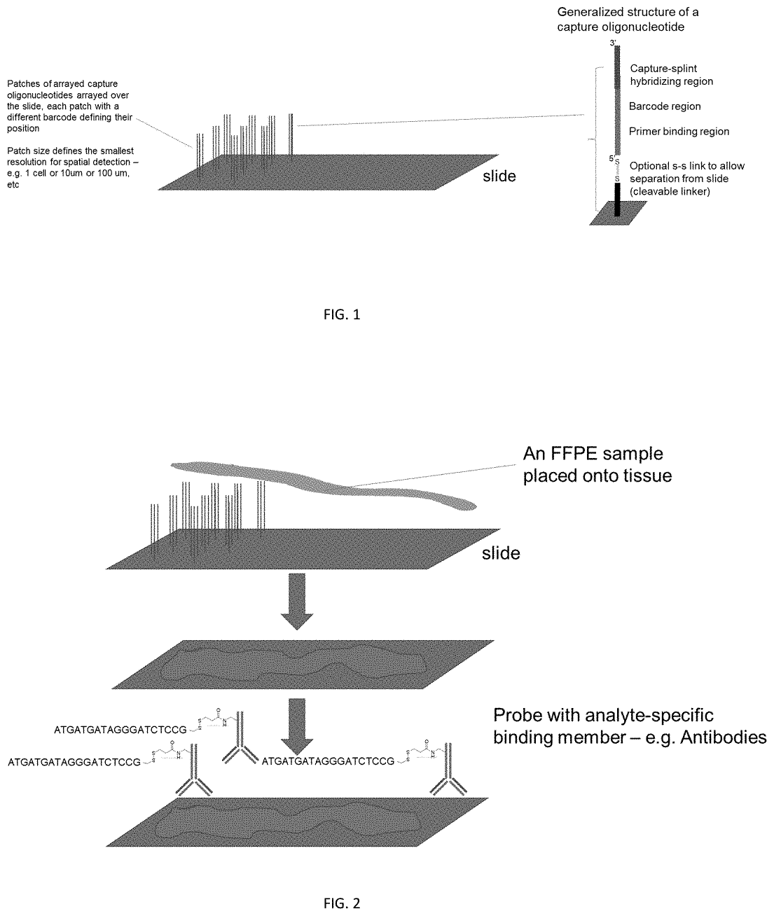

### DEFINITIONS

The term “nucleic acid” and “oligonucleotide” are used interchangeably herein to describe a polymer of any length. The length may vary as desired, ranging in some instances from 10 to 100,000, such as from 20 to 50,000 and including from 30 to 10,000 nucleotides. Oligonucleotides are usually synthetic and, in some embodiments, are under 120 nucleotides in length. An oligonucleotide may comprise deoxyribonucleotides or ribonucleotides, or compounds produced synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The terms “nucleoside” and “nucleotide” are intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

Unique Molecular Identifiers (i.e., UMIs) are randomers of varying length as desired, e.g., ranging in length in some instances from 6 to12 nucleotides, that can be used for counting of individual molecules of a given molecular species. Counting is achieved by attaching UMIs from a diverse pool of UMIs to individual molecules of a target of interest such that each individual molecule receives a unique UMI. By counting individual transcript molecules, PCR bias can be reduced during NGS library prep and a more quantitative understanding of the sample population can be achieved. See e.g., U.S. Pat. No. 8,835,358; Fu et al., “Molecular Indexing Enables Quantitative Targeted RNA Sequencing and Reveals Poor Efficiencies in Standard Library Preparations,” PNAS (2014) 5: 1891-1896 and Fu et al., “Digital Encoding of Cellular mRNAs Enabling Precise and Absolute Gene Expression Measurement by Single-Molecule Counting,” Anal. Chem (2014) 86:2867-2870.

By “sequencing platform adapter construct” is meant a nucleic acid construct that includes at least a portion of a nucleic acid domain (e.g., a sequencing platform adapter nucleic acid sequence) utilized by a sequencing platform of interest, such as a sequencing platform provided by Illumina® (e.g., the HiSeg™, MiSeg™ and/or Genome Analyzer™ sequencing systems); Ion Torrent™ (e.g., the Ion PGM™ and/or Ion Proton™ sequencing systems); Pacific Biosciences (e.g., the PACBIO RS II sequencing system); Life Technologies™ (e.g., a SOLiD sequencing system); Roche (e.g., the 454 GS FLX+ and/or GS Junior sequencing systems); or any other sequencing platform of interest.

In certain aspects, the sequencing platform adapter construct includes a nucleic acid domain selected from: a domain (e.g., a “capture site” or “capture sequence”) that specifically binds to a surface-attached sequencing platform oligonucleotide (e.g., the P5 or P7 oligonucleotides attached to the surface of a flow cell in an Illumina® sequencing system); a sequencing primer binding domain (e.g., a domain to which the Read 1 or Read 2 primers of the Illumina® platform may bind); a barcode domain (e.g., a domain that uniquely identifies the sample source of the nucleic acid being sequenced to enable sample multiplexing by marking every molecule from a given sample with a specific barcode or “tag”); a barcode sequencing primer binding domain (a domain to which a primer used for sequencing a barcode binds); a molecular identification domain (e.g., a molecular index tag, such as a randomized tag of 4, 6, or other number of nucleotides) for uniquely marking molecules of interest to determine expression levels based on the number of instances a unique tag is sequenced; or any combination of such domains. In certain aspects, a barcode domain (e.g., sample index tag) and a molecular identification domain (e.g., a molecular index tag) may be included in the same nucleic acid.

The sequencing platform adapter constructs may include nucleic acid domains (e.g., “sequencing adapters”) of any length and sequence suitable for the sequencing platform of interest. In certain aspects, the nucleic acid domains are from 4 to 200 nucleotides in length. For example, the nucleic acid domains may be from 4 to 100 nucleotides in length, such as from 6 to 75, from 8 to 50, or from 10 to 40 nucleotides in length. According to certain embodiments, the sequencing platform adapter construct includes a nucleic acid domain that is from 2 to 8 nucleotides in length, such as from 9 to 15, from 16-22, from 23-29, or from 30-36 nucleotides in length.

The nucleic acid domains may have a length and sequence that enables a polynucleotide (e.g., an oligonucleotide) employed by the sequencing platform of interest to specifically bind to the nucleic acid domain, e.g., for solid phase amplification and/or sequencing by synthesis of the cDNA insert flanked by the nucleic acid domains. Example nucleic acid domains include the P5 (5′-AATGATACGGCGACCACCGA-3′) (SEQ ID NO:01), P7 (5′-CAAGCAGAAGACGGCATACGAGAT-3′) (SEQ ID NO:02), Read 1 primer (5′-ACACTCTTTCCCTACACGACGCTCTTCCGATCT-3′) (SEQ ID NO:03) and Read 2 primer (5′-GTGACTGGAGTTCAGACGTGTGCTCTTCCGATCT-3′) (SEQ ID NO:04) domains employed on the Illumina®-based sequencing platforms. Other example nucleic acid domains include the A adapter (5′-CCATCTCATCCCTGCGTGTCTCCGACTCAG-3′) (SEQ ID NO:05) and P1 adapter (5′-CCTCTCTATGGGCAGTCGGTGAT-3′) (SEQ ID NO:06) domains employed on the Ion Torrent™-based sequencing platforms.

The nucleotide sequences of nucleic acid domains useful for sequencing on a sequencing platform of interest may vary and/or change over time. Adapter sequences are typically provided by the manufacturer of the sequencing platform (e.g., in technical documents provided with the sequencing system and/or available on the manufacturer's website). Based on such information, the sequence of the sequencing platform adapter construct of the template switch oligonucleotide (and optionally, a first strand synthesis primer, amplification primers, and/or the like) may be designed to include all or a portion of one or more nucleic acid domains in a configuration that enables sequencing the nucleic acid insert (corresponding to the template RNA) on the platform of interest.

The phrase “oligonucleotide indexed surface” refers to a surface on which oligonucleotides are immobilized on a substrate. The substrate can have a variety of configurations, e.g., a sheet, bead, or other structure. In certain embodiments, the collections of oligonucleotide probe elements employed herein are present on a surface of the same support, e.g., in the form of an array, such as a planar array of addressable oligonucleotide locations (e.g., spots), a bead array of beads in wells of a microwell array where the beads have oligonucleotides bound to them, etc.

The term “array” encompasses the term “microarray” and refers to an ordered array presented for binding to nucleic acids and the like.

An “array,” includes any two-dimensional or substantially two-dimensional (as well as a three-dimensional) arrangement of spatially addressable regions bearing nucleic acids, particularly oligonucleotides or synthetic mimetics thereof, and the like. Where the arrays are arrays of nucleic acids, the nucleic acids may be adsorbed, physisorbed, chemisorbed, or covalently attached to the arrays at any point or points along the nucleic acid chain.

As used herein, the term “hybridization” describes that a primer, or other polynucleotide, specifically hybridizes to a region of a target nucleic acid with which the primer or other polynucleotide shares some complementarity. Whether a primer specifically hybridizes to a target nucleic acid is determined by such factors as the degree of complementarity between the polymer and the target nucleic acid and the temperature at which the hybridization occurs, which may be informed by the melting temperature (Tₘ) of the primer. The melting temperature refers to the temperature at which half of the primer-target nucleic acid duplexes remain hybridized and half of the duplexes dissociate into single strands. The Tm of a duplex may be experimentally determined or predicted using the following formula Tm=81.5+16.6(log10[Na+])+0.41 (fraction G+C)−(60/N), where N is the chain length and [Na⁺] is less than 1 M. See Sambrook and Russell (2001; Molecular Cloning: A Laboratory Manual, 3rd ed., Cold Spring Harbor Press, Cold Spring Harbor N.Y., Ch. 10). Other more advanced models that depend on various parameters may also be used to predict Tm of primer/target duplexes depending on various hybridization conditions. Approaches for achieving specific nucleic acid hybridization may be found in, e.g., Tijssen, Laboratory Techniques in Biochemistry and Molecular Biology-Hybridization with Nucleic Acid Probes, part I, chapter 2, “Overview of principles of hybridization and the strategy of nucleic acid probe assays,” Elsevier (1993).

The terms “complementary” and “complementarity” as used herein refer to a nucleotide sequence that base-pairs by non-covalent bonds to all or a region of a target nucleic acid (e.g., a region of the product nucleic acid). In the canonical Watson-Crick base pairing, adenine (A) forms a base pair with thymine (T), as does guanine (G) with cytosine (C) in DNA. In RNA, thymine is replaced by uracil (U). As such, A is complementary to T and G is complementary to C. In RNA, A is complementary to U and vice versa. Typically, “complementary” refers to a nucleotide sequence that is at least partially complementary. The term “complementary” may also encompass duplexes that are fully complementary such that every nucleotide in one strand is complementary to every nucleotide in the other strand in corresponding positions. In certain cases, a nucleotide sequence may be partially complementary to a target, in which not all nucleotides are complementary to every nucleotide in the target nucleic acid in all the corresponding positions. For example, a primer may be perfectly (i.e., 100%) complementary to the target nucleic acid, or the primer and the target nucleic acid may share some degree of complementarity which is less than perfect (e.g., 70%, 75%, 85%, 90%, 95%, 99%).

The percent identity of two nucleotide sequences can be determined by aligning the sequences for optimal comparison purposes (e.g., gaps can be introduced in the sequence of a first sequence for optimal alignment). The nucleotides at corresponding positions are then compared, and the percent identity between the two sequences is a function of the number of identical positions shared by the sequences (i.e., % identity=# of identical positions/total # of positionsx100). When a position in one sequence is occupied by the same nucleotide as the corresponding position in the other sequence, then the molecules are identical at that position. A non-limiting example of such a mathematical algorithm is described in Karlin et al., Proc. Natl. Acad. Sci. USA 90:5873-5877 (1993). Such an algorithm is incorporated into the NBLAST and XBLAST programs (version 2.0) as described in Altschul et al., Nucleic Acids Res. 25:389-3402 (1997). When utilizing BLAST and Gapped BLAST programs, the default parameters of the respective programs (e.g., NBLAST) can be used. In one aspect, parameters for sequence comparison can be set at score=100, wordlength=12, or can be varied (e.g., wordlength=5 or wordlength=20).

The term “specific binding” refers to a direct association between two molecules, due to, for example, covalent, electrostatic, hydrophobic, and ionic and/or hydrogen-bond interactions, including interactions such as salt bridges and water bridges. An analyte-specific binding member and its corresponding analyte have binding specificity for one another. An analyte-specific binding member may be naturally derived or wholly or partially synthetically produced. An analyte-specific binding member has an area on its surface, or a cavity, which specifically binds to and is therefore complementary to a particular spatial and polar organization of the analyte. Thus, an analyte-specific binding member specifically binds to an analyte. Examples of pairs of analyte-specific binding members and analytes are antigen-antibody, biotin-avidin, hormone-hormone receptor, receptor-ligand, nucleic acids that hybridize with each other, and enzyme-substrate.

When an analyte-specific binding member comprising an analyte binding oligonucleotide, such oligonucleotide could be appended to the end of a detector oligonucleotide. Thus, an oligonucleotide could comprise a portion that is “a detector oligonucleotide” and a portion that is a “analyte binding oligonucleotide.” The analyte- binding oligonucleotide would recognize the analyte by hybridization. For example, an analyte binding oligonucleotide could specifically hybridize to a specific sequence in an mRNA, so that one can detect specific mRNAs by certain methods disclosed herein.

Analyte-specific binding members exhibit high affinity and binding specificity for the corresponding analyte. Typically, affinity between an analyte-specific binding member and its corresponding analyte is characterized by a K_{d} (dissociation constant) of 10⁻⁶ M or less, such as 10⁻⁷ M or less, including 10⁻⁸ M or less, e.g., 10⁻⁹ M or less, 10⁻¹⁰ M or less, 10⁻¹¹ M or less, 10⁻¹² M or less, 10⁻¹³ M or less, 10⁻¹⁴ M or less, including 10⁻¹⁵ M or less.

### DETAILED DESCRIPTION

The present disclosure provides methods for assessing a section of a biological sample (biological sample section), particularly, for determining the location of an analyte in the biological sample section. In an aspect, the present disclosure provides a method for assessing a biological sample section comprising: a) contacting the biological sample section with an oligonucleotide indexed surface comprising an addressable array of capture oligonucleotides; b) probing the oligonucleotide indexed surface contacted with the biological sample section with an analyte-specific binding member that specifically binds to the analyte, wherein the analyte-specific binding member comprises a detector oligonucleotide; c) linking the detector oligonucleotide to a capture oligonucleotide proximal thereto to produce a linked product nucleic acid, which linked product nucleic acid may be: i) a ligated product nucleic acid made up ligated detector and barcode capture oligonucleotides, or ii) an extension product nucleic acid produced by template mediated extension of hybridized detector and barcode capture oligonucleotides; and d) sequencing the linked product nucleic acid to assess the biological sample for the analyte. Also provided are kits and systems finding use in practicing various embodiments of the methods.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the methods, which are, for clarity, described in the context of separate embodiments, may also be provided in combination in a single embodiment. Conversely, various features of the methods, which are, for brevity, described in the context of a single embodiment, may also be provided separately or in any suitable sub-combination. All combinations of the embodiments are specifically embraced by the present invention and are disclosed herein just as if each and every combination was individually and explicitly disclosed, to the extent that such combinations embrace operable processes and/or devices/systems/kits. In addition, all sub-combinations listed in the embodiments describing such variables are also specifically embraced by the present methods and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### Methods

As summarized above, certain embodiments of the present disclosure provide a method of assessing a biological sample section for an analyte. The assessing can comprise determining the presence, quantity, and, where desired, location of an analyte in the biological sample section. According to certain embodiments, a three-dimensional biological sample can be divided in a plurality of sections and the location of an analyte can be assessed in the plurality of sections. The location of an analyte in the plurality of sections can be stacked to construct a three-dimensional representation of the biological sample along with the distribution of the analyte in the three-dimensional sample.

Such methods can also be practiced to determine desired spatial parameters of one or more analytes, such as the location of a plurality of analytes in a biological sample section, the relative relationships (i.e., positions) of a plurality of analytes with respect to each other, etc. The location of a plurality of analytes in a plurality of sample sections of a three-dimensional biological sample can be stacked to construct a three-dimensional representation of the biological sample along with the location of the plurality of analytes in the three-dimensional sample. The location of a plurality of analytes can be determined relative to each another. For example, when the location of a first analyte is known, the location of a second analyte can be determined relative to the first analyte. Such analysis would also help in determining relative positions of analytes when the position of the first analyte is known or determined.

The methods disclosed herein utilize an oligonucleotide indexed surface comprising capture oligonucleotides with known sequences and known locations on the indexed surface. A biological sample section is contacted with the capture oligonucleotide array, e.g., by laying the sample on to the array or the array on to the sample, and the section is then probed with an analyte-specific binding member that specifically binds to the analyte. The analyte-specific binding member comprises a detector oligonucleotide, the detector oligonucleotide having a known sequence specific to the analyte.

Binding of the analyte-specific binding member to the analyte in the biological sample section brings the detector oligonucleotide in proximity to a capture oligonucleotide of the array. The resultant proximal oligonucleotides may then be linked, e.g., as described below, to produce a linked product nucleic acid, which linked product nucleic acid may be: i) a ligated product nucleic acid made up ligated detector and barcode capture oligonucleotides or ii) an extension product nucleic acid produced by template mediated extension of hybridized detector and barcode capture oligonucleotides

As summarized above the proximal oligonucleotides may be linked using any different linking protocols. In some instances, a capture oligonucleotide and a proximally located detector oligonucleotide may be linked by ligation. Such ligation can be performed using any convenient protocol, e.g., a splint mediated ligation protocol or a non-splint mediated ligation protocol. In a splint mediated ligation protocol, a splint oligonucleotide having regions that hybridize with a region on the capture oligonucleotide and a region on the detector oligonucleotide may be employed to tie together a capture oligonucleotide and proximately located detector oligonucleotide. This action forms a double stranded structure comprising on one strand the splint oligonucleotide and on another strand the free end of the capture oligonucleotide and the free end of the detector oligonucleotide placed adjacent to each other. The adjacently placed ends of the capture oligonucleotide and the detector oligonucleotides are suitable for ligation via a ligase. In other instances, a splint is not employed, e.g., where proximal ends are ligated by a ligase that does not require a splint to maintain proximity of the ends. Any convenient ligase may be employed, where examples of ligases that may be employed include, but are not limited to: T4 DNA ligase, T3 DNA ligase, T7 DNA ligase, T4RNA ligase, Rtcb ligase, etc. Ligases of interest include those listed at the website having an address produced by placing “https://www.” before “neb.com/tools-and-resources/selection-charts/properties-of-dna-and-rna-ligases”.

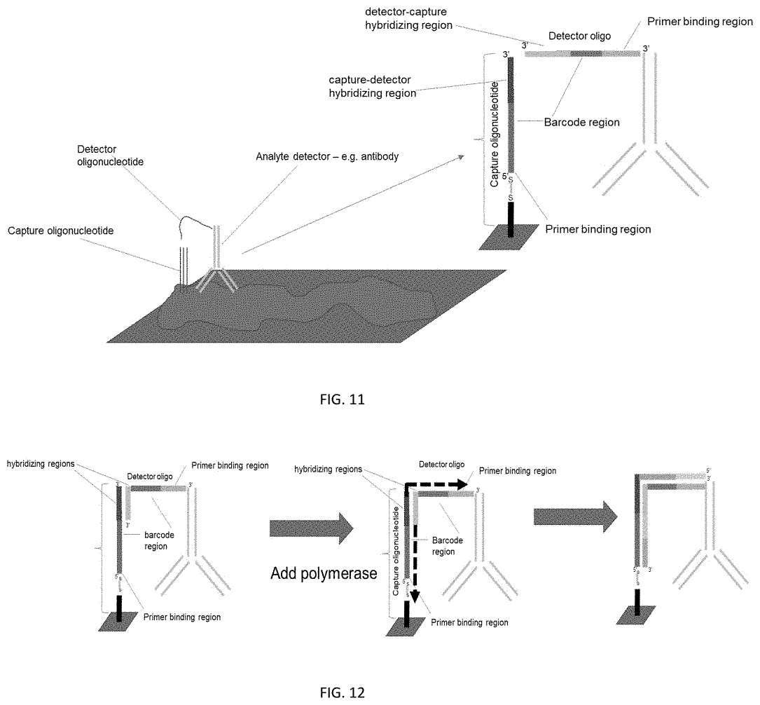

In yet other embodiments, linking is achieved via hybridization of complementary regions in the capture and detector oligonucleotides follow by production of a template mediated polymerization or extension produce nucleic acid to produce a product that can subsequently be sequenced. For example, a capture oligo and a detector oligo could each have a region that is complementary to the other. Therefore, a capture oligonucleotide would have a capture-detector hybridizing region, and a detector oligonucleotide would have a detector-capture hybridizing region. When in proximity, a capture oligonucleotide and a detector oligonucleotide would hybridize through these hybridizing regions, thereby producing a linked product nucleic acid. In the presence of a suitable polymerase, the ends of one or both oligonucleotides could be extended so as to copy the other oligonucleotide as a template. While the polymerase mediated extension could occur at the ends of both the capture and detector oligonucleotides, one of the oligonucleotides could be blocked from amplification. Such blocking could be achieved using any convenient blocker, such as 3′ NH₂ or other blocker (including those known in the art) on the last nucleotide to prevent extension. Thus, the polymerase mediated extension would happen on only the un-blocked oligonucleotide. The extension product nucleic acid can be further amplified and sequenced. Such template mediated extension produces an extension product nucleic acid having the sequences of both the capture oligonucleotide and the detector oligonucleotide. This derivative, or amplicon thereof, may then be sequenced. Certain such embodiments are described in FIGS. 11 and 12. Thus, the linkage of a capture oligonucleotide and a detector oligonucleotide can be used to obtain a linked product nucleic acid, which linked product nucleic acid may be: i) a ligated product nucleic acid made up ligated detector and barcode capture oligonucleotides or ii) an extension product nucleic acid produced by template mediated extension of hybridized detector and barcode capture oligonucleotides, that contains the information about the location of the capture oligonucleotide through a capture oligonucleotide specific barcode and the information about the analyte through the analyte-specific barcode of the detector oligonucleotide. Therefore, the sequence of the ligated product nucleic acid or template mediated polymerized derivative can be used to determine the location of an analyte on the indexed surface. This information can be further combined with an image of the biological sample section on the surface to determine the location of the analyte in the biological sample section.

Accordingly, certain embodiments of the invention provide a method of assessing a biological sample section for an analyte, the method comprising:

a) contacting the biological sample section with an oligonucleotide indexed surface comprising an addressable array of capture oligonucleotides;

b) probing the oligonucleotide indexed surface contacted with the biological sample section with an analyte-specific binding member that specifically binds to the analyte, wherein the analyte-specific binding member comprises a detector oligonucleotide;

c) linking the detector oligonucleotide to a barcoded capture oligonucleotide proximal thereto to produce a linked product nucleic acid, which linked product nucleic acid may be: i) a ligated product nucleic acid made up ligated detector and barcode capture oligonucleotides or ii) an extension product nucleic acid produced by template mediated extension of hybridized detector and barcode capture oligonucleotides; and

d) sequencing the linked product nucleic acid to assess the biological sample for the analyte.

A biological sample section can be a section routinely used in assessing biological samples. For example, the section can be a paraffin-embedded section or a frozen section. A paraffin-embedded section is typically produced by embedding a biological sample in a paraffin wax block. Thus, the tissue is enclosed in the wax block, which provides a framework for slicing the tissue. The paraffin wax block and, hence, the tissue, is sliced using a paraffin tissue slicer.

Sections having a thickness of between 5 μm and 20 μm are typically produced using a paraff in-embedded sectioning of a biological sample. A frozen section of a tissue can be produced by freezing a biological sample, for example, in liquid nitrogen. The frozen block of tissue is then mounted in a cryostat machine and is cut with a microtome. Sections having a thickness of as thin as 1 μm can be produced using frozen sectioning of a biological sample.

The thickness of the section may vary as desired. In some embodiments, the thickness of the biological sample section ranges from 1 μm to 20 μm, such as from 2 μm to 18 μm; from 3 μm to 16 μm; from 4 μm to 14 μm; from 5 μm to 12 μm; from 6 μm to 10 μm; or from 7 μm to 8 μm.

A biological sample section can be fixed in a fixative. Certain non-limiting examples of fixatives routine used in the art include formaldehyde, Bouin's fixative, Zenker's solution, Helly's solution, Carnoy's solution, acetone, methanol, ethanol, zinc formalin, and formaldehyde/glutaraldehyde solution, and combinations thereof. A person or ordinary skill in the art can select an appropriate fixative for use in the methods disclosed herein. Additional fixatives are also known in the art and can be used in the methods disclosed herein.

Where desired, the tissue section may be permeabilized, e.g., to provide access to analytes inside of cells. Permeabilization is optionally achieved without the addition of any exogenous protease. In such embodiments, permeabilization can be achieved by heating, and/or by addition of a permeabilizing agent. In one exemplary class of embodiments, the sample is incubated in a solution comprising a detergent or amphipathic glycoside at 0.01%-0.2% (v/v) prior to the probing, e.g., as described below. Suitable detergents and amphipathic glycosides are known in the art, and include, but are not limited to, saponin, Triton™X-100, digitonin, Leucoperm™, and Tween®20. The solution optionally also comprises other solvents and reagents, e.g., acetone, methanol, and/or formamide. In other embodiments, protease treatment that is gentler than that generally required with other techniques for in situ detection can be employed. For example, the sample can be incubated with proteinase K at a concentration of less than 1 ug/ml (e.g., 0.2-1 ug/ml or 20-100 ng/ml) prior to the hybridizing, binding, and detecting steps. Other suitable proteases are known in the art and can be employed in the methods, e.g., trypsin, pepsin, and protease type XIV. In one aspect, the samples are exposed only to a gentle pretreatment the cells or tissues are exposed to less than 1 ug/ml of protease at a temperature of 85° C. or less for 10 minutes or less. Optionally, the gentle pretreatment does not include exposure of the cells or tissues to an organic solvent (e.g., anhydrous) or exposure to an aldehyde (e.g., pretreatment without a cross-linking step). Further details regarding tissue sample section preparation are disclosed in U.S. Published Patent Application Publication Nos. 20160046984; 20140120534; and 20130004953; the disclosures of which are herein incorporated by reference.

As noted above, a capture oligonucleotide in the oligonucleotide indexed surface comprises a barcode unique to the location of the capture oligonucleotide. Capture oligonucleotides can further comprise one or more sequences that may perform one or more desired functions, such as to facilitate linking of the capture oligonucleotides to the detector oligonucleotides and/or sequencing the linked product nucleic acid, etc. For example, a capture oligonucleotide can comprise one or more of: a UMI, a capture-detector hybridizing region, a capture-primer hybridizing region, a capture-splint hybridizing region, a cleavable linker, a detectable label, and a sequencing platform adaptor construct. Where one or more of such domains are present, they may be arranged in any convenient order.

When present, the cleavable linker can facilitate releasing the linked product nucleic acid from the oligonucleotide indexed surface. The cleavable linker can be a photo-cleavable linker. A photocleavable linker connects the capture oligonucleotide to the surface and is photo-labile so that the linker can be cleaved using a light to release capture oligonucleotides from the surface. Typically, a photocleavable linker comprises a photo-labile bond that cleaves upon irradiation with a light, for example, a light of a specific wavelength. Certain examples of compounds containing photo-labile bonds include compounds containing groups selected from arylcarbonylmethyl, phenacyl, o-alklylphenacyl, p-hydroxyphenacyl, benzoin, o-nitrobenzyl, o-nitro-2-phenethyloxycarbonyl, o-nitroanilides, coumarin-4-ylmethyl, arylmethyl, o-hydroxyarylmethyl, pivaloyl, esters of carboxylic acids, arylsulfonyl, carbanion-mediated groups, 2-hydroxycinnamyl, α-keto amides, α,β-unsaturated anilides, methyl(phenyl)thiocarbamic acid, 2-pyrrolidino-1,4-benzoquinone, triazine, arylmethyleneimino, xanthene, pyronin, or a combination thereof. Certain specific examples of photo-labile compounds are disclosed in Klan et al. (2013), Chem. Rev. , 113, 1, 119-191, which is herein incorporated by reference in its entirety. Additional examples of photo-labile compounds are known in the art and can be used in the methods disclosed herein. The cleavable linker can also be a chemically cleavable linker. In one such example, the capture oligonucleotide is conjugated to the surface via a disulfide bond, which is cleaved using an oxidizing agent thereby releasing the capture oligonucleotides. In another example, the capture oligonucleotide is conjugated to the surface via a peptide, which can be cleaved using an enzyme thereby releasing the capture oligonucleotides. Enzymatically-cleavable linkers can comprise protease-sensitive amides or esters, P-lactamase-sensitive P-lactam analogs, thrombin cleavage sequences, enterokinase cleavage sequences, glycosidase-cleavable sugars, or a combination thereof. Chemically cleavable linkers can also comprise compounds containing groups selected from diol, diazo, esters, sulfones, diarylmethyl, trimethylarylmethyl, silyl esters, carbamates, oxyesters, thioesters, thionoesters, fluorinated amines, or combination thereof. Additional examples of chemically cleavable linkers are known in the art and can be used in the methods disclosed herein. The cleavable linker can also be thermally-cleavable. Examples of thermally cleavable linkers includes, but are not limited to: O-phenoxyacetyl; O- methoxyacetyl; O-acetyl; O-(p-toluene)sulfonate; O-phosphate; O-nitrate; O-[4-methoxy]- tetrahydrothiopyranyl; O-tetrahydrothiopyranyl; O-[5-methyl]-tetrahydrofuranyl; O-[2-methyl,4- methoxy]-tetrahydropyranyl; O-[5-methyl]-tetrahydropyranyl; and O-tetrahydrothiofuranyl. Further details regarding various types of cleavable linkers that may be employed in embodiments of the invention include those described in United States Published Application Publication No. 20190112648; the disclosure of which is herein incorporated by reference.

As noted above, each capture oligonucleotide comprises a barcode unique to the location of the capture oligonucleotide. The length of the unique barcodes can be varied as desired, ranging in some instances from four to twelve, such as from six to ten and including from seven to eight nucleotides. Table 1 below provides the number of random sequences produced for barcodes having different lengths:

| Number of nucleotides | Total number of possible |
|---|---|
| in a barcode | the barcode sequences |
| 4 | 256 |
| 5 | 1,024 |
| 6 | 4,096 |
| 7 | 16,384 |
| 8 | 65,536 |
| 9 | 262,144 |
| 10 | 1,048,576 |
| 11 | 4,194,304 |
| 12 | 16,777,216 |

In certain embodiments, the barcodes (in both capture and detector oligonucleotides) could be designed to permit error correction. Particularly, the barcodes can have a set Hamming distance between them, so that if there is a sequence error in the barcode, it can be corrected. (Hamming, R. W. (April 1950). “Error detecting and error correcting codes”. The Bell System Technical Journal. 29 (2): 147-160). For example, if all the barcodes differ by at least 3 nucleotides, then any single nucleotide change can be corrected back to the original correct sequence.

In addition to the unique barcode, a capture oligonucleotide can further comprise one or more of: a capture-detector hybridizing region, a UMI, a capture-primer hybridizing region, a capture-splint hybridizing region, a cleavable linker, a detectable label, and sequencing adaptor construct.

A capture-detector hybridizing region can be varied in length as desired, ranging in some instances from 4 to 35 nucleotides, such as from 4 to 10, 11 to 22 and including from 15 to 20 nucleotides. A capture-primer hybridizing region can be varied in length as desired, ranging in some instances from 10 to 25 nucleotides, such as from 12 to 22 and including from 15 to 20 nucleotides. Similarly, a capture-splint hybridizing region can be varied in length as desired, ranging in some instances from 5 to 35 nucleotides, such as 7 to 30 nucleotides, including 10 to 25 nucleotides, such as from 12 to 22 and including from 15 to 20 nucleotides. Accordingly, a capture oligonucleotide can be varied in length as desired, ranging in some instances from 25 to 200 nucleotides, such as 30 to 120 nucleotides, such as from 40 to 90, including from 50 to 80, and further including from 70 to 80 nucleotides.

A capture oligonucleotide can also comprise a detectable label, e.g., which renders the capture oligonucleotide array image addressable. A detectable label can be a fluorescent label, radio-label, or enzyme label. The fluorescent label used to detect the bead can be selected from a large number of dyes that are commercially available from a variety of sources, such as Molecular Probes (Eugene, OR) and Exciton (Dayton, OH). Examples of fluorophores of interest include, but are not limited to, 4-acetamido-4′-isothiocyanatostilbene -2,2′disulfonic acid; acridine and derivatives such as acridine, acridine orange, acridine yellow, acridine red, and acridine isothiocyanate; 5-(2′-aminoethyl)aminonaphthalene-1 -sulfonic acid (EDANS); 4-amino-N-[3-vinylsulfonyl)phenyl]naphthalimide-3,5 disulfonate (Lucifer Yellow VS); N-(4-anilino-1-naphthyl)maleimide; anthranilamide; Brilliant Yellow; coumarin and derivatives such as coumarin, 7-amino-4-methylcoumarin (AMC, Coumarin 120), 7-amino-4-trifluoromethylcouluarin (Coumarin 151); cyanine and derivatives such as cyanosine, Cy3, Cy5, Cy5.5, and Cy7; 4′,6-diaminidino-2-phenylindole (DAPI); 5′, 5″-dibromopyrogallol-sulfonephthalein (Bromopyrogallol Red); 7-diethylamino-3-(4′-isothiocyanatophenyl)-4-methylcoumarin; diethylaminocoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6-dichlorotriazin-2-yl)aminofluorescein (DTAF), 2′7′-dimethoxy-4′5′-dichloro-6-carboxyfluorescein

(JOE), fluorescein isothiocyanate (FITC), fluorescein chlorotriazinyl, naphthofluorescein, and QFITC (XRITC); fluorescamine; 1R144; IR1446; Green Fluorescent Protein (GFP); Reef Coral Fluorescent Protein (RCFP); Lissamine™; Lissamine rhodamine, Lucifer yellow; Malachite Green isothiocyanate; 4-methylumbelliferone; ortho cresolphthalein; nitrotyrosine; pararosaniline; Nile Red; Oregon Green; Phenol Red; B-phycoerythrin; o-phthaldialdehyde; pyrene and derivatives such as pyrene, pyrene butyrate and succinimidyl 1-pyrene butyrate; Reactive Red 4 (Cibacron™ Brilliant Red 3B-A); rhodamine and derivatives such as 6-carboxy-X-rhodamine (ROX), 6-carboxyrhodamine (R6G), 4,7-dichlororhodamine lissamine, rhodamine B sulfonyl chloride, rhodamine (Rhod), rhodamine B, rhodamine 123, rhodamine X isothiocyanate, sulforhodamine B, sulforhodamine 101, sulfonyl chloride derivative of sulforhodamine 101 (Texas Red), N,N,N′,N′-tetramethyl-6-carboxyrhodamine (TAMRA), tetramethyl rhodamine, and tetramethyl rhodamine isothiocyanate (TRITC); riboflavin; rosolic acid and terbium chelate derivatives; xanthene; or combinations thereof. Other fluorophores or combinations thereof known to those skilled in the art may also be used, for example those available from Molecular Probes (Eugene, Oreg.) and Exciton (Dayton, Ohio).

As summarized above, the capture oligonucleotide is present on an oligonucleotide indexed surface comprising an addressable array of oligonucleotides. An array is “addressable” when it has multiple regions of different moieties (e.g., different capture oligonucleotides) such that a spot at a particular predetermined location (i.e., an “address”) on the array will contain a capture oligonucleotide having a particular sequence. Array features are typically, but need not be, separated by intervening spaces. In some cases, the addressable array of capture oligonucleotides comprises unique spots of capture oligonucleotides. With advances in the relevant technology, a large number of unique oligonucleotides can be deposited in small spots on a surface. Accordingly, an addressable array of capture oligonucleotides can contain unique spots of capture oligonucleotides in a number that varies as desired, where in some instances the number ranges from: 150 to 50,000; 500 to 30,000; 1000 to 25,000; 2000 to 20,000; 3000 to 15,000; or 5000 to 10,000. The spots can be of any shape, such as a circle, square, ellipse, or oval. The longest dimension of the spot can vary as desired, and in some instances is equal to or less than: 200 μm, 175 μm, 150 μm, 125 μm, 100 μm, 75 μm, 50 μm, 25 μm, 20 μm, 15 μm, 10 μm, 5 μm, 2 μm, or 1 μm. Accordingly, an addressable array of capture oligonucleotides can have any density of unique spots of capture oligonucleotides as desired, where in some instances the density ranges from 2000 to 4 million per square centimeter. In some cases, the density of unique spots of capture oligonucleotides can be more than 4 million per square centimeter, or as suitable for a particular method. The addressable array of capture oligonucleotides can be of any size. In some cases, the addressable array of capture oligonucleotides has dimensions selected from the group consisting of: 75 mm×25 mm, 75×50 mm, 46×27 mm, 48×28 mm, and 18×18 mm.

The oligonucleotide indexed surface can be made from a solid support material. Such material can be selected from any suitable material, where materials that may be employed include glass, nitrocellulose, silicon, plastic, and combinations thereof. In certain embodiments, the oligonucleotide indexed surface comprises a solid support of plastic, such as polystyrene, polycarbonate, polyvinyl chloride, polypropylene, and combinations thereof.

A substrate may carry one, two, four or more arrays disposed on a front surface of the substrate. Depending upon the use, any or all of the arrays may be the same or different from one another and each may contain multiple spots.

Addressable arrays employed in embodiments of the invention may be prepared using any convenient protocol. In some instances, preparation of addressable array of capture oligonucleotides comprises obtaining of the sequences, selection and preparation of suitable surface, and depositing the oligonucleotides on its surface. The oligonucleotide array can be made by chemically synthesizing the oligonucleotides on a surface. The oligonucleotide array can also be made by attaching pre-made oligonucleotides to the surface. Typically, this is done by photolithography, mechanical micro spotting, or inkjets. Certain details of the process that can be used to prepare an addressable array of capture oligonucleotides are provided in Fesseha et al. (2020), Pathol Lab Med Open J.; 1(1): 54-62, which is herein incorporated by reference in its entirety. For example, arrays can be fabricated using drop deposition from pulse-jets of either nucleic acid precursor units (such as monomers) in the case of in situ fabrication, or the previously obtained nucleic acid. Such methods are described in detail in, for example, the previously cited references including U.S. Pat. No. 6,242,266, U.S. Pat. No. 6,232,072, U.S. Pat. No. 6,180,351, U.S. Pat. No. 6,171,797, and U.S. Pat. No. 6,323,043, and the references cited therein. As already mentioned, these references are incorporated herein by reference. Other drop deposition methods can be used for fabrication, as previously described herein. Also, instead of drop deposition methods, photolithographic array fabrication methods may be used. Inter-feature areas need not be present particularly when the arrays are made by photolithographic methods as described in those patents.

A solid support can be functionalized with a functional group, which facilitates conjugating the solid support to the oligonucleotides. Particularly, oligonucleotides are deposited onto the functionalized surface and functional groups on the 5′ or 3′ end of the oligonucleotide reacts with the functional group on the surface to covalently attach the oligonucleotide to the surface. For example, an amino group on an oligonucleotide can conjugate to a group on the surface selected from isothiocyanate, carbon disulfide, and sulfonyl chloride. Additional examples of chemistry used to conjugate oligonucleotides to a solid surface are known in the art and can be used in the methods disclosed herein.

Arrays that may be employed in embodiments of the invention also include random arrays, in which the identity of a probe cannot be determined from its location. An example of a random array is ILLUMINA®BEADARRAY™ in which individual reactive microbeads are randomly placed into wells etched on a microwell array plate. The identity of a probe in random arrays may be determined using bead encoding and subsequent decoding, i.e. each bead carries a unique identifying label. A variety of bead encoding technologies are known in the art. In such arrays, “bead” and “microbead”, which are used interchangeably, may refer to a microparticle that is approximately spherical and has a diameter greater than approximately 1 micron and smaller than approximately 1 mm. It should be however understood that beads smaller than 1 micron, for example 100 nm or 500 nm and beads larger than 1 mm, for example 2 mm or 5 mm may also be used in some embodiments. Furthermore, microparticles that are not spherical, e.g. microrods or microcubes, microparticles that have irregular shape and microparticles that have cavities may be also used in some embodiments of the instant disclosure. For non-spherical microparticles, the size of the microparticle may be estimated based on its largest linear dimension. For microparticles that expand their volume when exposed to a particular solvent, the size of the microparticle may be provided for the dry form, as well as the swollen form. The beads are placed into wells of a microwell plate. The terms “microwell array plate” and “microwell plate”, may refer to a three-dimensional solid support comprising a plurality of microwells. The term “microwell” may refer to a topological feature such as a well, a pit, a depression and similar, in which at least one of the linear dimensions is greater than 1 micron but smaller than 1 mm. In some embodiments, wells with linear dimensions greater than 1 mm may be also referred to as microwells, for example in reference to the industry-standard 96-well or 384-well plates. Bead arrays finding use in embodiments of the invention are further described in U.S. Pat. Nos. 10,101,336 and 9,611,507; the disclosures of which with respect to bead arrays are incorporated herein by reference. Use of bead arrays for spatial transcriptomic analysis have been described in Vickovic, S., et al. High-definition spatial transcriptomics for in situ tissue profiling. Nat Methods 16, 987-990 (2019).

As reviewed above, aspects of the methods include contacting the biological sample section with an oligonucleotide indexed surface comprising an addressable array of capture oligonucleotides. In some instances, the step of contacting the biological sample section with the oligonucleotide indexed surface comprises laying the biological sample section onto the oligonucleotide indexed surface. The biological sample section can be imaged on the indexed surface, which can be used to determine the location of an analyte in the section. Alternatively, second oligonucleotide indexed surface can be laid onto the tissue sample section. In certain embodiments, a second oligonucleotide indexed surface is laid onto the biological sample section laid onto the oligonucleotide indexed surface, wherein the second oligonucleotide indexed surface comprises a second addressable array of capture oligonucleotides. Thus, the biological sample section is sandwiched between the two arrays in which capture oligonucleotides are on both surfaces of the biological sample section. In this embodiment, two unique oligonucleotides are present at any given position on the biological sample section, thus increasing the chances and accuracy of assessing the analyte in the biological sample section. Note that by using two oligonucleotide indexed surfaces, these may also be positioned such that the locations of the oligonucleotides alternate in space. This provides benefit in that generally oligonucleotide array surfaces have spots of oligos separated by an area that is not spotted—e.g. spots can be 10, 50, 100 uM apart. Thus, areas of the section to be analyzed will not be in contact with an oligo nucleotide spot. However, if a second array is employed simultaneously that has its register shifted as compared to the first so that the spots of oligonucleotides on the second array fall in the locations of the gaps between the spots on the first array, more of the sample's surface area will be in contact with oligonucleotide spots and therefore be available for analysis.

An analyte that can be assessed according to the methods disclosed herein can be any biological entity of interest, where examples of analytes that may be assessed using methods of the invention include, but are not limited to, proteins, nucleic acids, e.g., DNA, RNA, lipids, or carbohydrates. Similarly, the analyte-specific binding member can vary, and in some instances may be an antibody, oligonucleotide, aptamer, polypeptide, carbohydrate, lipid, or small molecule that specifically binds to the analyte.

As noted above, the analyte-specific binding member comprises a detector oligonucleotide. In certain embodiments, the detector oligonucleotide comprises a barcode unique to the analyte. The unique barcode in the detector oligonucleotide, when associated with a unique capture oligonucleotide on the array, e.g., via linking or template mediated polymerization extension, captures the information about the location of the analyte on the array and the presence of the analyte in the corresponding position. The unique barcode in the detector oligonucleotide can be varied in length as desired, ranging in some instances from four to twelve, such as from six to ten, including from seven to eight nucleotides. Table 1 above provides the number of random sequences produced for tags having different lengths. In certain embodiments, the unique barcodes used in the capture oligonucleotides are not used in the detector oligonucleotides, i.e., a group of unique sequences is used in capture oligonucleotides and a different group of unique sequences is used in the detector oligonucleotides. Where desired, the detector barcodes may employ Hamming distance error correction.

In addition to the unique barcode, the detector oligonucleotide may include one or more additional functional domains or elements, (in any desired order) such as but not limited to: a detector-primer hybridizing region; a UMI, a detector-capture hybridizing region, a detector-splint hybridizing region; a detectable label, a cleavable linker, a sequencing platform adaptor construct, etc. When present, a detector-capture hybridizing region can be varied in length as desired, ranging in some instances from 5 to 35 nucleotides, such as 7 to 30 nucleotides, including 10 to 25 nucleotides, such as from 12 to 22 and including from 15 to 20 nucleotides. Also, when present, a detector-primer hybridizing region can be varied in length as desired, ranging in some instances from 10 to 25 nucleotides, such as from 12 to 22 and including from 15 to 20 nucleotides. Similarly, when present, a detector-splint hybridizing region can vary be varied in length, ranging in some instances 5 to 35 nucleotides, such as 7 to 30 nucleotides, including from 10 to 25 nucleotides, such as from 12 to 22 and including from 15 to 20 nucleotides. Accordingly, a detector oligonucleotide can be of any desired length, in some instances, range in length from 30 to 120, such as from 40 to 90, and including from 50 to 80 or 60 to 70 nucleotides.

The detector oligonucleotide can be conjugated to the analyte-specific binding member via a linker. In certain embodiments, the linker is a photo-cleavable linker, a chemically cleavable linker, or a thermally cleavable linker. Any of the linkers discussed above in connection with the capture oligonucleotides can be used in the detector oligonucleotides. When the analyte-specific binding member is an oligonucleotide comprising an analyte-binding oligonucleotide and a detector oligonucleotide, a linker connects the analyte binding oligonucleotide with the detector oligonucleotide. In such cases, the linker connects the two oligonucleotides, the analyte binding oligonucleotide and the detector oligonucleotide. In such cases the linker maybe an oligo nucleotide and maybe as little as a phosphodiester bond between one oligo and the next.

As summarized above, aspects of the invention include probing the oligonucleotide indexed surface contacted with the biological sample section with an analyte-specific binding member that specifically binds to the analyte of interest. Probing the oligonucleotide indexed surface contacted biological sample section with the analyte-specific binding member may include incubating the biological sample section with the analyte-specific binding member for duration and under conditions that allow binding of the analyte-specific binding member to the analyte. The conditions and duration that allow binding of the analyte-specific binding member to the analyte depend on the analyte and the binding member and can be determined on the case by case basis. These conditions include appropriate temperature, pH, buffer, etc. A person of ordinary skill in the art can determine appropriate conditions for a particular pair of analyte and analyte-specific binding member. After probing the biological sample section with an analyte-specific binding member, the biological sample section can be washed to remove any unbound analyte-specific binding member. Conditions for such washing also depend on the analyte and the binding member and can be determined on the case by case basis. Thus, a person of ordinary skill in the art can determine appropriate washing conditions for a particular pair of analyte and analyte-specific binding member. After the washing step, the oligonucleotide indexed surface comprises the capture oligonucleotides, biological sample section laid on top of it, and analyte-specific binding members bound to the corresponding analytes. Thus, the detector oligonucleotides on the analyte-specific binding members are held in proximity to the capture oligonucleotides.

In one embodiment, the capture oligonucleotide and the detector oligonucleotide are linked via the capture-detector hybridizing region and the detector-capture hybridizing region. When in proximity, these regions hybridize with each other to form a double stranded oligonucleotide with single stranded overhangs, the double stranded region made from the capture-detector hybridizing region and the detector-capture hybridizing region hybridized with each other. Moreover, at the point of the overhang, the ends of the hybridizing regions can provide extension sites for the oligonucleotides.

Therefore, in the presence of a polymerase, the free ends of one or both hybridizing oligonucleotides could be used as primers and could be extended to produce a copy of the other oligonucleotide, using the other oligonucleotide as a template, so producing an extension product nucleic acid having the sequence of the detector oligonucleotide and a sequence complementary to the sequence of the capture oligonucleotide. An of producing such extended oligonucleotides is shown in FIGS. 11 and 12.

As shown in FIGS. 11 and 12, both the capture and detector oligonucleotides could be extended. In certain embodiments, extension from one of the oligonucleotides could be blocked. Such blockage can be provided by introducing a 3′NH₂ group on one of the oligonucleotides. Thus, polymerase extension could only occur on the unmodified oligonucleotide. Each extended product, i.e., extension product nucleic acid, contains a barcode or its complement from a capture oligonucleotide, which provides the information about the location, and a barcode or its complement from a detector oligonucleotide, which provides the information about the analyte. Based on the presence in an extended product of a barcode or its complement specific to a location and a barcode or its complement specific to an analyte, one can determine the presence of the analyte at the position of the capture oligonucleotide. Because the extension product nucleic acid contains the sequence information for both a capture oligonucleotide and a proximally located detector oligonucleotide, such extended product nucleic acid is also considered herein to be a linked product nucleic acid.

In certain embodiments, linking a capture oligonucleotide to a detector oligonucleotide can be done by ligating the proximally located oligonucleotides. In some cases, such methods include ligating proximal detector and capture oligonucleotides. The proximately located capture oligonucleotides and detector oligonucleotides may be ligated using any convenient protocol. In some instances, ligating a detector oligonucleotide to a capture oligonucleotide proximal thereto to produce the ligated product nucleic acid can be performed using splint mediate ligation protocol. In such splint mediated ligation protocols, a splint oligonucleotide that hybridizes to a capture oligonucleotide and a detector oligonucleotide is employed. In some instances, a splint oligonucleotide comprises a capture hybridizing region that is complementary to the capture-splint hybridizing region on the capture oligonucleotide and a detector hybridizing region that is complementary to the detector splint hybridizing region. The capture hybridizing region can vary in length as desired, ranging in some instances from between 10 and 20 nucleotides. The detector hybridizing region can also vary in length as desired, ranging in some instances from between 10 and 20 nucleotides. The splint oligonucleotide can also vary in length as desired, and in some instances, can be between 20 and 40 nucleotides in length.

In splint mediated ligation protocols, ligating a detector oligonucleotide to a proximately located barcoded capture oligonucleotide is performed by hybridizing a splint oligonucleotide to:

1) a capture-splint hybridizing region on the capture oligonucleotide via a capture hybridizing region on the splint oligonucleotide that is complementary to capture-splint hybridizing region and 2) a detector-splint hybridizing region on the detector oligonucleotide via a detector hybridizing region on the splint oligonucleotide that is complementary to the detector-splint hybridizing region. Thus, a double stranded oligonucleotide is produced, which comprises, as one strand, the splint oligonucleotide and, as the other strand, capture-splint hybridizing region and detector-splint hybridizing region separated by a nick. This nick can be sealed using a ligase. Thus, the method further comprises ligating via a ligase the capture-splint hybridizing region of the capture oligonucleotide and the detector-splint hybridizing region of the detector oligonucleotide to produce a ligated product nucleic acid. Again, the production of the ligated product nucleic acid produces a snapshot of the location on the indexed array and the presence of an analyte at that location.

To further assess the biological sample section, the linked product nucleic acids produced after the linking step can be sequenced. Such sequencing can be performed using any convenient sequencing protocol. In some instances, sequencing is performed by amplifying, e.g., via a polymerase chain reaction, the linked product nucleic acid, e.g., the ligation production nucleic acid or template dependent polymerase derivative, to produce amplicons and then sequencing the resultant amplicons. When present, the capture-primer hybridizing region and detector-primer hybridizing region can facilitate such sequencing. For example, PCR can be performed using a capture-primer and/or a detector-primer to produce multiple copies of the linked product nucleic acids. In some instances, both the capture primer and detector primer are used for PCR amplification; however, linked product nucleic acid can be amplified using only one of these primers. Also, the same capture primer sequence can be used in the capture oligonucleotides and the same detector primer sequence can be used in the detector oligonucleotides. Thus, one primer or one primer pair could be used to amplify all of the linked product nucleic acids. The PCR amplification can be performed while the linked product nucleic acids are attached to the oligonucleotide indexed surface. The PCR amplification can also be performed after the linked product nucleic acids are cleaved from the oligonucleotide indexed surface, for example, using the photo-cleavable, chemically-cleavable, enzymatically cleavable, e.g., via restriction enzyme or a cas9/CRISPR system or other appropriate enzymatic method such as a peptidase, hydrolase, etc., or thermally-cleavable linkers present in the capture oligonucleotides and/or detector oligonucleotides. If the linked product nucleic acids are cleaved from the indexed surface, before PCR amplification, the nucleic acids can be purified from the remnants of the biological sample section.

Sequencing of the linked product nucleic acid or amplicons produced therefrom, e.g., as described above, may be performed using any convenient sequencing protocol. The PCR amplified copies of the linked product nucleic acids can be sequenced, for example, using high-throughput sequencing, such as a next generation sequencing. The next generation sequencing can be any convenient sequencing protocol, such as but not limited to paired-end sequencing, ion-proton sequencing, pyrosequencing, nanopore sequencing. A person of ordinary skill in the art can readily identify and use appropriate sequencing methods to determine the sequences of the linked product nucleic acids.

The methods disclosed herein can also be used to assess the biological sample section for a plurality of analytes. Accordingly, in certain embodiments, an oligonucleotide indexed surface contacted biological sample section can be probed with, in addition to the analyte-specific binding member, one or more additional analyte-specific binding members. While the number of different analytes that may be assessed in such instances may vary, in some such instances the number ranges from 2 to 20,000, such as 2 to 10, 000, e.g., 2 to 1,000, where in some instances the number ranges from 2 to 500, e.g., 5 to 100. Each additional analyte-specific binding member specifically can bind to an additional analyte. Also, each of the additional analyte-specific binding members can comprise an additional detector oligonucleotide and the additional detector oligonucleotide can comprise a barcode unique to the additional analyte. Thus, the methods disclosed herein can be multiplexed to assess the biological sample section with multiple analytes. Like the analyte, the one or more additional analytes can vary, where in some instances the one or more additional analytes is a protein, DNA, RNA, lipid, or carbohydrate. Similarly, like the analyte-specific binding member, the one or more additional analyte-specific binding members can, in some instances, be an antibody, oligonucleotide, aptamer, polypeptide, carbohydrate, lipid, or small molecule. The biological sample section can be contacted simultaneously with the analyte-specific binding member and the one or more additional analyte-specific binding members. This can be done when all of the analyte-specific binding members bind to their corresponding analytes under same or similar conditions. The biological sample section can be contacted sequentially with the analyte-specific binding member and the one or more additional analyte-specific binding members. This can be done when different analyte-specific binding members require different conditions for binding to their corresponding analytes. After the one or more additional analyte-specific binding members bind to their corresponding analytes, the biological sample section can be washed to remove any unbound analyte-specific binding members. Appropriate conditions for washing can be readily determined by a person of ordinary skill in the art depending on the analyte-specific binding members and the corresponding analytes. Similar to the linking step disclosed above for the capture oligonucleotide and a detector oligonucleotide, capture oligonucleotides from the indexed array can be linked with detector oligonucleotides from the one or more additional analyte-specific binding members. Linking the capture oligonucleotides with detector oligonucleotides from the one or more additional analyte-specific binding members would produce one or more additional linked product nucleic acids. The one or more additional linked product nucleic acids can be amplified and sequenced, similar to the linked product nucleic acid discussed above. The same detector primer sequence can be used in the one or more additional detector oligonucleotides. Thus, one primer or one primer pair could be used to amplify all of the linked product nucleic acids. Sequencing of the linked product nucleic acid and/or the one or more additional linked product nucleic acids provides sequencing data that can be used to assess the analyte and/or one or more additional analytes in the biological sample section. For example, presence in a linked nucleic acid of a barcode unique to a capture oligonucleotide known to be present a specific location on the indexed array and another barcode unique to an analyte-specific binding member indicates that the analyte was located in the biological sample section at the site of the specific capture oligonucleotide. This, when combined with the image of the biological sample section, can be used to determine the location of the analyte within the biological sample section. Thus, the methods disclosed herein could be used to determine the locations of a plurality of analytes in a biological sample section based on the sequencing data of the linked product nucleic acids and without the need for imaging specific for the analytes.

In certain embodiments, the information about the location of one more analytes in two-dimensional section can be extrapolated to determine the location of the one or more analytes in a three-dimensional biological sample. For example, a three-dimensional biological sample can be sliced into a plurality of sections and the location of one or more analytes can be determined in the plurality of sections using the methods disclosed herein. The information about the location of the one or more analytes in the plurality of sections can be compiled to determine the location of the one or more analytes in the three-dimensional biological sample.

In certain embodiments, the location of the one or more analytes in the two-dimensional or the three-dimensional biological sample could be used to evaluate the sample, for example, for histology, pathology, or morphology of the biological sample. For example, the presence of a particular analyte at specific location in the biological sample may be indicative of the presence of a disease. Thus, the methods disclosed herein may be used for diagnostic purposes, particularly, pathological diagnosis, etc.

### Kits

Aspects of the present disclosure also include kits. The kits could be used to carry out the methods of the invention. Thus, the kits may include one or more of: a) an oligonucleotide indexed surface comprising an addressable array of capture oligonucleotides; and b) an analyte-specific binding member that specifically binds to the analyte, wherein the analyte-specific binding member comprises a detector oligonucleotide, e.g., as described above, or one or more reagents for producing analyte-specific binding members, such as detector oligonucleotides, which may be functionalized for ready conjugation to an analyte binder, e.g., via any convenient conjugation protocol (e.g., thiol linking moieties, “Click chemistry” linking moieties, etc.). The kits can further include reagents, e.g., polymerases, buffers, controls (positive and/or negative), containers, and instructions necessary to carry out the methods disclosed herein.

The details described above in the methods of the invention, for example, regarding the capture oligonucleotides, indexed array, analyte-specific binding member, detector oligonucleotides, the analytes, etc., are also applicable to the kits disclosed herein and such embodiments are within the purview of the invention. Certain such details are described in Clauses 57-82 provided below.

Certain embodiments of the invention also provide an oligonucleotide indexed surface comprising an addressable array of capture oligonucleotides. The details described above in the methods of the invention regarding the oligonucleotide indexed surface are also applicable to the oligonucleotide indexed surface envisioned herein and such embodiments are within the purview of the invention. Certain such details are described in Clauses 83-95 provided below.

Further embodiments of the invention provide an analyte-specific binding member that specifically binds to the analyte, wherein the analyte-specific binding member comprises an analyte-specific binding domain and a detector oligonucleotide. The details described above in the methods of the invention regarding the analyte-specific binding members are also applicable to the analyte-specific binding member envisioned herein and such embodiments are within the purview of the invention. Certain such details are described in Clauses 96-104 provided below.

In addition to the above-mentioned components, a subject kit may further include instructions for using the components of the kit, e.g., to practice the subject methods as described above. The instructions are generally recorded on a suitable recording medium. The instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g. CD-ROM, diskette, Hard Disk Drive (HDD), portable flash drive, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### Computer Systems

The present disclosure provides computer control systems that are programmed to implement methods of the disclosure. For example, a computer system can be configured to collect images of a biological sample (e.g., an FFPE sample) before and/or after the sample is sectioned for further processing. The system can store and process microscopic images of different tissue sections of the sample to obtain genomics data from each section and then to collectively process data from all the sections using suitable algorithms. The system can obtain spatial information of a region of the sample before and/or after it is probed using an arrayed slide by determining the X/Y coordinates of different regions of a section. In addition, the system can assess histology/pathology of the sample, evaluate morphology or assess any other features of the sample. Further, the system can record any signal from detector labels (e.g., fluorescent label) the detector oligonucleotides and/or analyte detector (e.g., antibody) may have. The system can further correlate the signal from the detector oligo and/or analyte detector with the region directly on the biological sample in question, (optionally histology stained) or be used to correlate between immediately preceding or subsequent FFPE sections i.e. typically in wax embedding methodologies (the predominant format) lie withinl0 microns sections slices. Once the library is generated and sequenced using sequencing primers, the computer system can parse the sequencing data to generate genomics data, such as transcriptomics, proteomics, and determine the proximity of different analytes, e.g., RNA and proteins, in the sample. A major feature of the proposed imaging system is to stack serial images recreating a cross-sectional, three-dimensional spatial reconstruction of the tissue, permitting analysis not only within a single plane (typically 10 microns) but across multiple sectioned planes within the sample. Based on the resultant spatial reconstruction, the system can provide information on the degree of analyte presence (ie T cell, or tumor cell infiltration) permitting a graded understanding biological/clinical significance of various analytes.

Accordingly, aspects of the invention further include systems, e.g., computer-based systems, which are configured to assess a biological sample section, particularly, to determine the location of an analyte in a biological sample section, e.g., as described above. A “computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based systems are suitable for use in the present invention. The data storage means may comprise any manufacture comprising a recording of the present information as described above, or a memory access means that can access such a manufacture.

To “record” data, programming or other information on a computer readable medium refers to a process for storing information, using any such methods as known in the art. Any convenient data storage structure may be chosen, based on the means used to access the stored information. A variety of data processor programs and formats can be used for storage, e.g., word processing text file, database format, etc.

A “processor” references any hardware and/or software combination that will perform the functions required of it. For example, any processor herein may be a programmable digital microprocessor such as available in the form of an electronic controller, mainframe, server or personal computer (desktop or portable). Where the processor is programmable, suitable programming can be communicated from a remote location to the processor, or previously saved in a computer program product (such as a portable or fixed computer readable storage medium, whether magnetic, optical or solid- state device based). For example, a magnetic medium or optical disk may carry the programming, and can be read by a suitable reader communicating with each processor at its corresponding station.

Embodiments of the subject systems include the following components: (a) a communications module for facilitating information transfer between the system and one or more users, e.g., via a user computer, as described below; and (b) a processing module for performing one or more tasks involved in the quantitative analysis methods of the invention.

In certain embodiments, a computer program product is described comprising a computer usable medium having control logic (computer software program, including program code) stored therein. The control logic, when executed by the processor of the computer, causes the processor to perform functions described herein. In other embodiments, some functions are implemented primarily in hardware using, for example, a hardware state machine. Implementation of the hardware state machine so as to perform the functions described herein may be accomplished using any convenient method and techniques.

In addition to the sensor device and signal processing module, e.g., as described above, systems of the invention may include a number of additional components, such as data output devices, e.g., monitors and/or speakers, data input devices, e.g., interface ports, keyboards, etc., fluid handling components, power sources, etc.

Accordingly, certain embodiments of the invention provide a system for assessing a biological sample section. Such system is configured to receive and process data to analyze a biological sample section. In some embodiments, such system comprises a processing module configured to receive the following data: i) an image of a biological sample section, ii) sequences of capture oligonucleotides in an addressable array of capture oligonucleotides, wherein each capture oligonucleotide comprises a barcode unique to the location of the capture oligonucleotide on the array, and iii) sequences of linked product nucleic acids obtained by processing the biological sample section according the methods disclosed herein, wherein the processing module is configured to process the received data to determine the location of the analyte in the biological sample section. Systems may be configured to receive information on the barcodes of the detectors used, e.g., in the form of a table associating barcodes to the analytes examined, so that the system can assign the analytes to the locations based on the barcodes of the detector and the capture oligo found in the product nucleic acids

For example, the system is configured to analyze the sequence of a linked product nucleic acid comprising a barcode from a capture oligonucleotide and a barcode from a detector oligonucleotide. The system is configured to then assign the analyte as identified by the barcode from the detector oligonucleotide to the location on the oligonucleotide indexed surface based on the barcode unique to the capture oligonucleotide. The system can be configured to assign the analyte on the entirety of the oligonucleotide indexed surface to determine the location of the analyte on the surface. The system can further be configured to compare the image of the biological sample section placed on the oligonucleotide indexed surface with the assigned location of the analyte on the oligonucleotide indexed surface to determine the location of the analyte within the biological sample section.

In addition to identifying the location of an analyte in a biological sample, the number of copies of the detector barcodes found at a location could be used to estimate the amount of the analyte that was present at that location. Thus, the number of copies of detector barcodes found at a location could provide quantitative information about an analyte. Such quantitative estimation can be further facilitated if the capture and/or the detector oligonucleotides contain UMIs. The UMI analysis could further enhance the analysis by providing a more digital estimate of amount that would correct for noise introduced by the PCR amplification.

In certain embodiments, the system comprises a processing module configured to receive the following data: i) a plurality of images of a plurality of sections of a three-dimensional biological sample, ii) sequences of capture oligonucleotides in a plurality of addressable arrays of capture oligonucleotides, wherein each capture oligonucleotide comprises a barcode unique to the location of the capture oligonucleotide on the plurality of arrays, iii) information on the barcodes of the detectors used, e.g., in the form of a table associating barcodes to the analytes examined, and iv)sequences of linked product nucleic acid and/or additional linked product nucleic acids obtained by processing the plurality of sections of the three-dimensional biological sample according the methods disclosed herein, wherein the processing module is configured to process the received data to determine the location of the analyte and/or the one or more additional analytes in the three-dimensional biological sample by stacking the determined location of the analyte and/or the one or more additional analytes in the plurality of sections of the three-dimensional biological sample.

Here, the system can be configured to stack the location of one or more analytes in a plurality of biological sample sections to determine the location of the one or more analytes within a three-dimensional biological sample.

The system can further be configured to evaluate the sample, for example, for histology, pathology, or morphology of the biological sample. For example, the system can be configured to determine, based on the presence of a particular analyte at specific location in the biological sample, the presence of a particular disease. Thus, the systems disclosed herein could be used for diagnostic purposes, particularly, pathological diagnosis.

### Utility

Assessment of a biological sample section as disclosed herein can find use in a variety of applications. Such assessments are useful in acquiring sample data and/or cellular data that may be used in making determinations pertaining to the sample and/or the cells of the sample. Samples that may be assessed according to the methods described herein have been described in detail above and generally include laboratory research samples (e.g., those described for non-clinical research use), clinical research samples, patient samples, diagnostic samples, prognostic samples, treatment samples, and the like. The combined data of the described sample evaluations are useful in comparing samples to one another, e.g., as in the comparison of two research samples (e.g., two research samples treated with two different experimental agents), comparison of an experimental sample to a control (e.g., comparison of a treated sample to an untreated control) and in comparing samples to a reference (e.g., a control reference or a reference value such as a healthy sample or diseased sample).

As described above, assessments based on the collected data find use in pathological diagnosis and in making assessments of whether a subject has a disease.

The assessments described herein find use in screening of subjects for disease, either as a first-line of detection in suspected healthy individuals and/or as a surveillance mechanism for those at increased risk of developing a disease. The assessments may be performed independently or combined with conventional routine screening and biopsy advancing the rate of detection, reducing the rate of false negative and false positive assessments, and generally improving the standard of care related disease detection, monitoring, and treatment.

In addition to the above described patient assessments, the assessments described also find use in the research setting in evaluation of obtained sample data from laboratory, pre-clinical, and clinical models. For example, owing to the biological nature of the described methods, samples may be assayed directly from a host animal and evaluated according to the methods described herein.

In some embodiments, the methods disclosed herein find use in clinical practice. For example, the methods may be used to assess the effect of a drug on disease regression and monitoring treatment as well as in a trial to test a drug's efficacy. For example, distribution of an analyte in a biological sample before and after a drug treatment may be compared to determine the effect of the drug on the biological sample.

Furthermore, given the non-subjective and unbiased approach of the assessments described herein, the described methods also find use in combination with clinical research, e.g., in evaluating pre- and post-treatment samples for treatment effectiveness and/or monitoring treatment effectiveness during testing through one or more assessments during the course of a clinical trial.

The methods disclosed herein find use in the assessment of a broad variety of biological samples, for example, clinical specimens. Such clinical specimens include tissue samples obtained from any tissue to be assessed including placenta, brain, eyes, pineal gland, pituitary gland, thyroid gland, parathyroid glands, thorax, heart, lung, esophagus, thymus gland, pleura, adrenal glands, appendix, gall bladder, urinary bladder, large intestine, small intestine, kidney, liver, pancreas, spleen, stoma, ovary, uterus, testis, and skin, to name a few.

The above described uses are in no way to be considered limiting as the methods and systems described herein may have additional utility not described herein.

The following example(s) is/are offered by way of illustration and not by way of limitation.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, cells, and kits for methods referred to in, or related to, this disclosure are available from commercial vendors such as BioRad, Agilent Technologies, Thermo Fisher Scientific, Sigma-Aldrich, New England Biolabs (NEB), Takara Bio USA, Inc., and the like, as well as repositories such as e.g., Addgene, Inc., American Type Culture Collection (ATCC), and the like.

### Example 1

### Oligonucleotide Indexed Array to Simultaneously Determine the Location of a Protein and RNA in a Biological Sample Section

This Example addresses some of these problems in the relevant art, particularly cost and complexity while potentially enabling simultaneous or substantially simultaneous analysis of both protein and RNA. Since the method is not constrained by visual resolution (an issue faced by SeqFISH, MERFISH and others), it could enable analysis of a greater number of analytes simultaneously. The equipment required for the users is also relatively uncomplicated. Both the equipment and workflow itself are relatively unsophisticated, thereby imparting a degree of robustness and subsequently general utility for both research and commercial users.

This Example provides the use an oligo indexed surface, such as a microscope slide which is modified, as necessary, permitting it to be coated with an addressable (optionally image addressable) array of barcoded oligonucleotides. In some cases, the surface can be a glass slide or some other suitable surfaces. The oligonucleotides are placed on the surface in an orderly array with different barcodes, so that the barcode defines the spatial location of the oligo on the surface. In addition to the barcode sequence, the oligonucleotides encode at least a primer binding region for amplification and a splint compatible region to promote proximity ligation to the detector oligonucleotides as will be described below. A variety of methods for making arrays of oligonucleotides on slides, such as Clontech's former Atlas Glass array products or other arrayed oligo systems for Gene expression analysis or SNP detection may be used.

Onto slides or surfaces are placed a section of tissue, for example, a section of FFPE tissue i.e., 1 slide per section. This can be prepared as is typically performed for Immunohistochemistry or alternatively as described for Ligation in situ hybridization (LISH: doi: 10.1093/nar/gkx471). The sample can then be either probed with antibodies (Ab) or hybridized with nucleic-acid type probes to mRNA (or other RNA species of interest). This could be performed using an Ab, nucleic-acid type probes or as a combination of the two. Note that antibodies are just one example of a suitable analyte detection reagent. Essentially, any analyte detection reagent could be used in place of, or in addition to, an antibody—e.g. an aptamer or a ligand for a receptor or any protein, carbohydrate, lipid or small molecule that specifically interacts with an analyte of interest. Again, the researcher could probe with any one of these analyte binding classes of molecules or a combination of these either sequentially or in parallel. Moreover, for each analyte type, the researcher may probe with one member of a class or many classes. For example, we envisage probing with an Ab targeting a specific or semi-specific epitope or 2 Abs directed to different proteins. Multiple Abs—e.g. 2-10, 10- to 100 or 1000 may be simultaneously or substantially simultaneously employed, as appropriate for the question asked. Similarly, the researcher could use one of any other class of analyte detection reagent or more than one, such as 2-10, 10- to 100 or 1000, or more as the researcher desires. We note the 10× spatial profiling technology cannot work with FFPE-type samples, which limits that applications generic utility, since most archival specimens are FFPE rather than frozen sections.

Major characteristics regarding these analyte-specific reagents are described below. Firstly, these reagents can be covalently linked to a detector oligonucleotide. Secondly, the detector oligonucleotide can include at least a barcode sequence, which determines the identity of the targeted analyte. Consequently, by determining the barcode sequence (or color moiety) it is possible to know which analyte was detected. Thirdly, the reagents can encompass a primer binding sequence permitting nucleic acid amplification. Fourthly, the reagents can bear a ligation splint region, promoting proximity ligation to the spatial positioned barcodes “pre-arrayed” on the slide. Detector oligonucleotides may optionally include other sequences as necessary—e.g. UMIs.

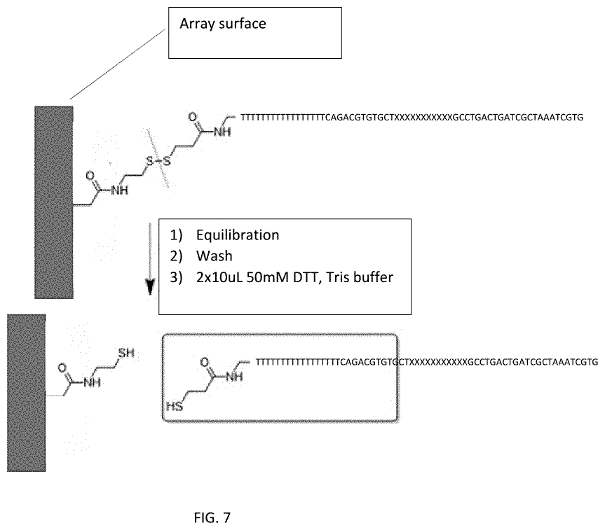

Once bound to the tissue sample, excess/unbound analyte detectors may be removed by washing, for example. The slide can then be treated with a ligation mixture comprising a ligase and a splint connector oligo which may comprise at least two hybridizing regions. The first region being complementary to the splint hybridizing region of the capture oligonucleotide and the second region may be complementary to the hybridizing region on the detector oligo. This generates a double stranded region that brings together barcode and detector oligonucleotides that are close together in space. Ligase present in the mixture can then be able to ligate these oligonucleotides together to form a single ligated product nucleic acid. This principal is described in: Cytokine detection by antibody-based proximity ligation (Mats Gullberg, et al.; PNAS June 1, 2004 101 (22) 8420-8424; world-wide-website: //doi.org/10.1073/pnas.0400552101). After ligation, PCR can be performed on the ligated molecules using primers corresponding to the two respective primer binding regions—either directly on the slide, or after first dissociating the oligonucleotides from the slide. This dissociation can be accomplished, for example, by using disulfide linkages that can be reduced to thiols to release the oligonucleotide as shown in FIG. 7.

In some cases, a sample can be probed with a first set of one or more (including hundreds or thousands) of different analyte detector reagents and a ligation step to link the analyte detector oligonucleotides to capture oligonucleotides can be performed. The analyte detectors can be removed, leaving behind ligated oligonucleotides. This may be performed by any convenient means, for example, by including disulfide links between the detector oligo and the analyte, so that the detector oligo can be separated from the detector reagent by exposure to reducing conditions to break the disulfide bonds. Alternatively, the use of a photo-cleavable -labile linker to generate a spatially localized, ligation competent phosphate group is envisaged. See Glen Research or Gene link synthesis world-wide-website:

genelink.com/newsite/products/modoligosPHOTOCLEAVABLE.asp

Once unwanted probes (e.g. one or more analyte detector reagents) are removed, the process may be repeated with fresh probes, so that serial rounds of probe addition, ligation and probe removal can occur. This approach is recommended if the probes require different conditions for binding to their cognate analytes (e.g. probe first with Ab against protein analytes and then probe with DNA oligo probes against specific mRNAs in order to obtain combined protein and DNA information). It could also be done to avoid steric interference between probes if the researcher desires to use many at once. When performing such serial reactions, each round of detector oligonucleotides could be ligated to remaining free capture oligonucleotides on the surface of the slide. Alternatively, each round of detector oligonucleotides could be ligated to the newly released free end of the previous detector oligonucleotide. So, for example, in a first round, the first detector oligo can be ligated to a capture oligonucleotide on the surface. Then in the next round, the second detector oligonucleotide can be ligated to the free end of the first detector oligonucleotide, so that now both detector oligonucleotides are ligated in series to the same capture oligonucleotide. This could be repeated as many times as needed, so generating a string of detector oligonucleotides ligated to the same capture oligonucleotide. The above protocol may be modified as desired where ligation is not employed to produce the linked product nucleic acid. For example, in protocols where linkage includes hybridization of detector and capture oligonucleotides to produce an extension product nucleic acid, in each round the prior extension product nucleic acid can be considered as a capture oligonucleotide for the next round of linking. In such embodiments, all that is necessary is that the oligonucleotides are configured such that each new free end provided in a given extension product nucleic acid has an appropriate detector oligonucleotide hybridizing region so that it can be linked to the next added detector oligonucleotide. Regardless of which approached is employed, all the barcodes could then be read in a sequencing reaction. If this approach is used, the PCR amplification sequence could be omitted from the detector oligonucleotides except for the last one of the series. Such analysis would also help in determining relative positions of analytes when the position of the first analyte is known or determined.

After dissociation, the oligonucleotides can be pooled, concentrated by methods well known in the art, resuspended a small volume (e.g. 25-50 μl) and PCR amplified by standard techniques. If PCR is performed directly on the surface without dissociation this can be done in ways known in the art such as bridge amplification as used in Illumina sequencing or PCR on slides for in situ hybridization (for example, see: world-wide-website: bio-rad.com/en-us/faq/900060/per-cycling-on-a-microscope-slide).

For PCR amplification, primers that either partially (bear positional or sequencer flow cell compatible tags) or entirely hybridize to the primer binding sites can be used. Examples include, but not limited to, sequences necessary to enable high throughput sequencing—e.g. Illumina sequencing. Thus, they may include Read primer 1 and/or 2 sequence and p5 and/or p7 sequences as well as index regions to allow pooling of multiple sequences. Alternatively, some of these sequences might be included in the spatial index and/or detect oligo sequences as appropriate, depending on the specific experiment or interest of the researcher—whatever is most convenient.

Experimental setup of the above-described disclosure may be presented in various embodiments. For example, while the assay workflow remains the same, an oligo indexed slide could directly be mounted onto an FFPE slide, instead of placing the FFPE sample onto the oligo slide. In addition, the size of an oligo indexed slide could vary in size, so the entire or portions of the FFPE slides are assayed.

In another embodiment, a second slide with capture oligonucleotides (or surface) can be brought in on top of a slide (surface) on which the tissue sample was placed, so that that both surfaces have capture oligonucleotides—thus making a sandwich in which capture oligonucleotides are both on the surface below the tissue sample and above. Thus, effectively doubling the number of capture oligonucleotides available at any given X/Y position on the slide for ligation to the detector oligonucleotides.

### Example 2

### Biotinylation of acGFP (Aequorea coerulescens GFP) Can Be Biotinylated

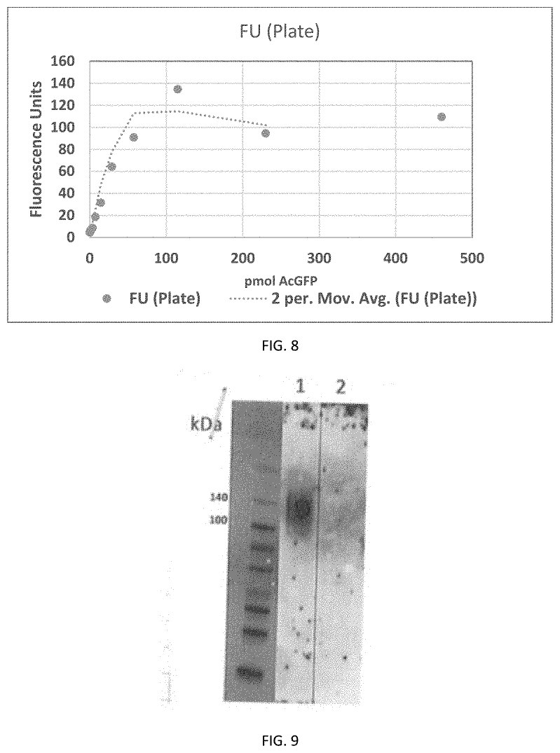

AcGFP, analyte, was biotinylated using the ChromaLink® Biotin Protein Labeling Kit (B-9007-105K, TriLink Biotechnologies), following the manufacturer's instructions and tested for binding to the wells of a streptavidin-coated 96-well plate. After washing the unbound material using a wash buffer, the fluorescence signal due to bound acGFP was measured using a plate reader. As shown in FIG. 8, specific binding of biotinylated AcGFP to the plate was observed, saturating at around 120 μmol; the expected capacity of the plate.

### Example 3

### Conjugation of Analyte-Specific Detector Oligonucleotide to Anti-AcGFP Antibody (JL-List of Sequences Used in the Examples 3-5:

Detector oligonucleotide (AF Oligo 3)
SEQ ID NO: 06
/5Phos/TCG TGT CTA ATA TNN NNN NNN NNN NNN NNC CTC
CGA AGG AAG ATC GGA AGA GCG TCG TGT AGG GTT TTT
TTT TT/3ThioMC3-D/
Capture oligonucleotide (AF Oligo 1)
SEQ ID NO: 07
/5BiotinTEG/TTT TTT TTT TTT TTT TTC AGA CGT GTG
CTC TTC CGA TCT AAC TTA TAC GGG ACG AAC CGC TTT
GCC TGA CTG ATC GCT AAA TCG TG
Splint Oligonucleotide (AF Oligo 4)
SEQ ID NO: 08
5′-TAC TTA GAC ACG ACA CGA TTT AGT TT/3AmMO/-3′
Fwd PCR primer:
SEQ ID NO: 09
TTCAGACGTGTGCTCTTCCGA
Rev PCR primer:
SEQ ID NO: 10
ACCCTACACGACGCTCTT
Synthetic positive control template:
SEQ ID NO: 11
TTC AGA CGT GTG CTC TTC CGA TCT AAC TTA TAC GGG
ACG AAC CGC TTT GCC TGA CTG ATC GCT AAA TCG TG TCG
TGT CTA ATA TNN NNN NNN NNN NNN NNC CTC CGA AGG
AAG ATC GGA AGA GCG TCG TGT AGG GT

Takara Bio USA's CapturemTM Protein G Mini prep columns were customized and used for the synthesis of an analyte-specific binding member, i.e., anti-AcGFP antibody in this case, conjugated to a detector oligonucleotide. Detector oligonucleotides were synthesized with thiol modification at the 3′ end and free 5′ phosphorylated end. The thiol was then reduced using TCEP*HCl (Aldrich- C4706-2G) reducing agent. The reduced 3′ end of the detector oligonucleotide was linked to analyte-specific binding member, in this case monoclonal antibodies, or anti-AcGFP antibody, using heterobifunctional crosslinker N-succinimidyl-3-(2-pyridyldithio) propionate (SPDP) (Thermo Scientific, Cat #21857). To demonstrate the presence of the conjugates, a biotinylated detector oligonucleotide conjugated to an anti-AcGFP antibody was used in a Western Blot procedure. As a control, a non-biotinylated detector oligonucleotide conjugated to an anti-AcGFP antibody was used. As shown in FIG. 9, the presence was confirmed of biotinylated detector oligonucleotide, in turn, confirming the presence of an anti-AcGFP antibody conjugated to the biotinylated detector oligonucleotide. The band at about 140 kDa in Lane 1 shows biotinylated AF3-olionucleotide (detector oligo) conjugated to JL-8 antibody while the reaction with JL-8 antibody (analyte-specific binding member) conjugated to AF3 oligonucleotide (SEQ ID NO: 06) did not produce a band in Lane 2.

### Example 4

### Proximity Ligation Assay (PLA) Between Surface-Bound Capture Oligonucleotide and Detector Oligonucleotide

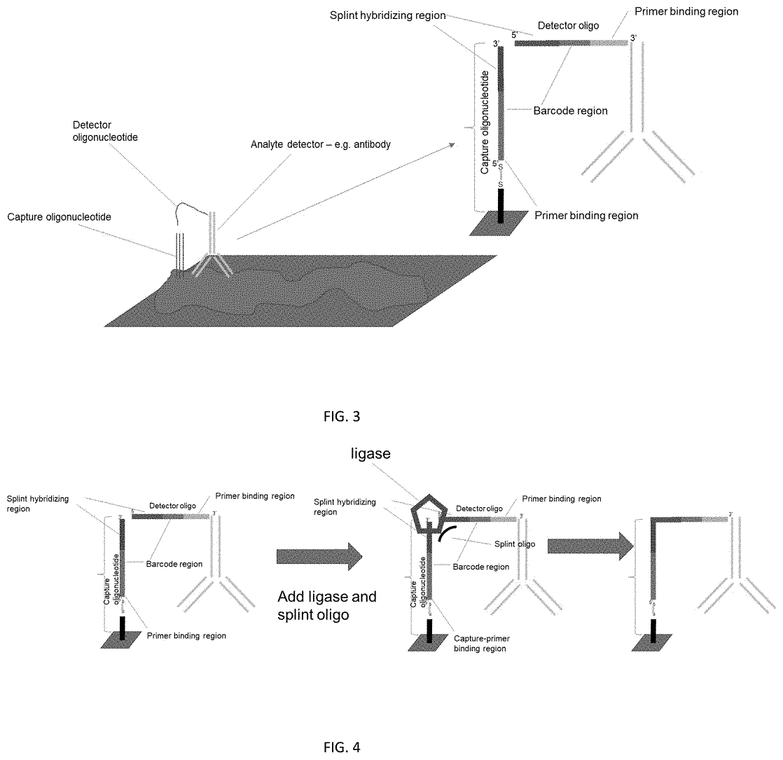

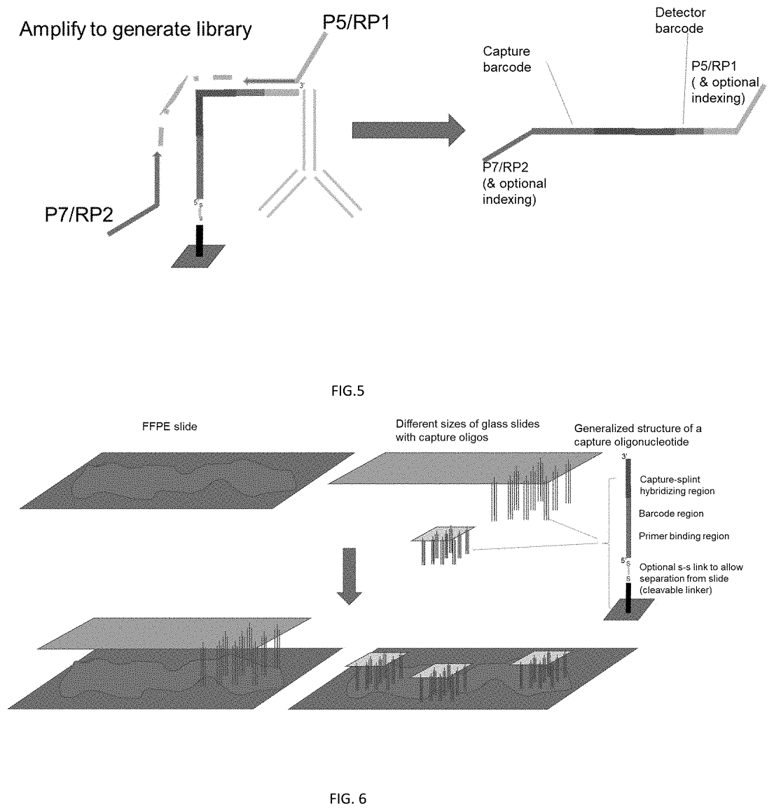

As a proof-of-principle for the spatial barcoding by proximity ligation assay, the following experiment was performed. The capture oligonucleotides were allowed to bind to the surface of a streptavidin-coated PCR tube (SW400021- Arctic white (Biomat.it)), so as to mimic the presence of a surface bound capture oligonucleotide in close proximity with an analyte protein (in this case biotinylated AcGFP) from an applied tissue sample (e.g. as depicted in FIG. 3). After binding, the tube was washed to remove unbound material and then probed with the analyte-specific anti-AcGFP antibody, conjugated to the analyte-specific detector oligonucleotide. The tubes were then washed again, and ligase and splint oligonucleotide were added. Finally, a PCR reaction mix was added to the tubes and the tubes were subjected to qPCR. As for controls, the reaction was performed in the absence of either the capture oligonucleotide, the analyte-specific binding member conjugated to the detector oligonucleotide, ligase, or the splint oligonucleotide. An outline of the protocol steps is as follows:
1. Streptavidin coated PCR strip tubes (SW400021- Arctic white (Biomat.it), were washed twice with 200 μl wash buffer (Tris-buffered saline (25 mM Tris,150 mM NaCl; pH 7.2), 0.1% BSA, 0.05% Tween20 detergent).
2. Tubes were incubated at room temperature (RT) for 1 hr with 1 μl of 30 fmol analyte protein (biotinylated AcGFP) and 1 μl of 30 fmol capture oligonucleotide in 100 μl 1× PBS buffer as detailed in Table 2 below.
3. Tubes were washed twice with 200 μl wash buffer followed by incubation at RT for 1 hr with 6 μmol of anti-AcGFP antibody conjugated to the analyte-specific detector oligonucleotide in 100 μl 1× Low TE buffer (pH 8.0, 10 mM Tris base, 0.1 mM EDTA) as detailed in Table 2 below.
4. Tubes were washed twice with 200 μl wash buffer and were incubated at RT for 10 mins with 0.8 U (0.2 μl) ligase (T4 DNA Ligase, M0202S, NEB) and 1 μl of 30 fmol splint oligonucleotide diluted in low TE buffer in 100 μl of ligase buffer (10 μl of 10×) and 88.8 μl of water followed by washing twice with 200 μl water.
5. Quantitative PCR was performed using 100 μl of TB Green® Advantage® qPCR Premix (Cat. No. 639676, Takara Bio USA). For the positive control reactions (Reactions 6-9), a synthetic template (SEQ ID: 11) was added to the TB Green® Advantage® qPCR Premix and subjected to qPCR.

As shown Table 2, Reactions 2-5 had the Ct for the amplification similar to the non-template control reaction, Reaction 9 (i.e., around Ct=30). In contrast, the complete reaction mix (Reaction 1), which included both the capture oligonucleotide and the analyte-specific binding member conjugated to the detector oligonucleotide, generated a positive Ct of 21, similar to a 1 pg input of the synthetic product of the ligation reaction used as a positive control (Reaction 7).

The results of this experiment are detailed in Table 2 below:

TABLE 2

| Reaction | | | |
|---|---|---|---|
| number | Reactions | Mean Ct | Tm |
| 1 | 30 fmol AcGFP + 30 fmol | 21.1 | 81.8 |
| | capture oligonucleotide (SEQ ID: | | |
| | 07) + 6 pmol anti-AcGFP antibody + | | |
| | 0.8 U ligase + 30 fmol | | |
| | splint oligonucleotide (SEQ ID: 08) | | |
| 2 | 30 fmol AcGFP + No capture | 30.2 | 82.4 |
| | oligonucleotide + 6 pmol | | |
| | Anti-AcGFP antibody + 0.8 U | | |
| | ligase + 30 fmol splint | | |
| | oligonucleotide (SEQ ID: 08) | | |
| 3 | 30 fmol AcGFP + 30 fmol | 33.9 | 83.4 |
| | capture oligonucleotide (SEQ ID: | | |
| | 07) + No Anti-AcGFP antibody + | | |
| | 0.8 U ligase + 30 fmol | | |
| | splint oligonucleotide (SEQ ID: 08) | | |
| 4 | 30 fmol AcGFP + 30 fmol | 29.1 | 82.3 |
| | capture oligonucleotide (SEQ ID: | | |
| | 07) + 6 pmol Anti-AcGFP antibody + | | |
| | No ligase + 30 fmol | | |
| | splint oligonucleotide (SEQ ID: 08) | | |
| 5 | 30 fmol AcGFP + 30 fmol capture | 28.5 | 81.6 |
| | oligonucleotide (SEQ ID: | | |
| | 07) + 6 pmol Anti-AcGFP antibody + | | |
| | 0.8 U ligase + No splint | | |
| | oligonucleotide | | |
| 6 | Positive control (SEQ ID: 11) 100 pg | 10.2 | 81.8 |
| 7 | Positive control (SEQ ID: 11) 1 pg | 20.3 | 81.9 |
| 8 | Positive control (SEQ ID: 11) 10 fg | 26.4 | 81.9 |
| 9 | No template control (NTC) | 33.9 | 83.6 |

### Example 5

### Confirmation that the PCR Reaction is Specific

In this Example, it is demonstrated that the PCR product produced from the ligated capture oligonucleotide and detector oligonucleotide is specific by showing that amplification requires both the fwd PCR primer (SEQ ID: 09, which hybridizes to a region on the spatial-positioning barcode oligonucleotide) and the rev PCR primer (SEQ ID: 10, which binds to a region on the detector oligonucleotide). The PLA reaction was performed as described above in Example 4. However, at the PCR step, either both fwd and rev primers were included (Reaction 1) or only one of the primers (fwd per primer or rev per primer) was included. For example, the fwd per primer (SEQ ID: 09) was included in Reaction 2 while rev per primer (SEQ ID: 10) was included in Reaction 3, as shown in Table 3.

Table 3 shows that only Reaction 1 resulted in a detectable PCR product.

| Reaction | Sample Name | Ct | Tm |
|---|---|---|---|
| 1 | Analyte + capture oligonucleotide (SEQ ID: | 18.2 | 81.3 |
| | 07) + detector oligonucleotide (SEQ ID: 06) + | | |
| | Ligase + Splint oligonucleotide (SEQ ID: | | |
| | 08) → PCR with fwd and | | |
| | rev PCR primers (SEQ IDs: 09-10) | | |
| 2 | Analyte + capture oligonucleotide (SEQ ID: | Undetermined | |
| | 07) + detector oligonucleotide (SEQ ID: 06) + | | |
| | Ligase + Splint oligonucleotide (SEQ ID: | | |
| | 08) → PCR with fwd PCR primer (SEQ ID: | | |
| | 09) | | |
| 3 | Analyte + capture oligonucleotide (SEQ ID: | Undetermined | |
| | 07) + detector oligonucleotide (SEQ ID: 06) + | | |
| | Ligase + Splint oligonucleotide (SEQ ID: | | |
| | 08) PCR with rev PCR primer (SEQ ID: | | |
| | 10) | | |

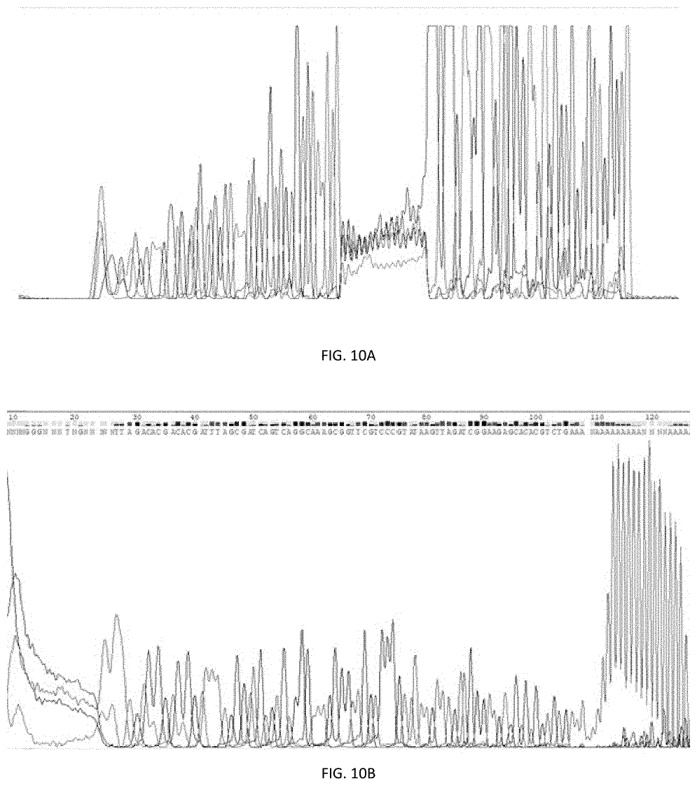

The ligated product of Reaction 1 was sequenced using both the fwd and reverse primers and the chromatograms for these sequencing reactions are provided in FIGS. 10A-10B. These confirmed that the expected product was generated and comprised of the capture oligonucleotide ligated to the detector oligonucleotide. Thus confirming that by reading this sequence, it was confirmed that the analyte (AcGFP) and the capture oligo were in close proximity on the surface of the tube.

Notwithstanding the appended claims, the disclosure is also defined by the following clauses:

Clause 1. A method of assessing a biological sample section for an analyte, the method comprising:
a) contacting the biological sample section with an oligonucleotide indexed surface comprising an addressable array of capture oligonucleotides;
b) probing the oligonucleotide indexed surface contacted with the biological sample section with an analyte-specific binding member that specifically binds to the analyte, wherein the analyte-specific binding member comprises a detector oligonucleotide;
c) linking the detector oligonucleotide to a barcoded capture oligonucleotide proximal thereto to produce a linked product nucleic acid, which linked product nucleic acid may be:
i) a ligated product nucleic acid made up ligated detector and barcode capture oligonucleotides; or
ii) an extension product nucleic acid produced by template mediated extension of hybridized detector and barcode capture oligonucleotides; and
d) sequencing the linked product nucleic acid to assess the biological sample for the analyte.

Clause 2. The method of clause 1, wherein the biological sample section is a paraffin-embedded section or a frozen section.

Clause 3. The method of clause 2, wherein the biological sample section is a paraffin-embedded section, and the paraffin-embedded section is fixed with a fixative.

Clause 4. The method of clause 3, wherein the fixative is selected from the group consisting of formaldehyde, Bouin's fixative, Zenker's solution, Helly's solution, Carnoy's solution, acetone, methanol, ethanol, zinc formalin, formaldehyde/glutaraldehyde solution, and combinations thereof.

Clause 5. The method of any of the preceding clauses, wherein the thickness of the biological sample section ranges from 1 μm to 20 μm.

Clause 6. The method of clause 5, wherein the thickness of the biological sample section ranges from 2 μm to 18 μm.

Clause 7. The method of any of preceding clauses, wherein each capture oligonucleotide in the oligonucleotide indexed surface comprises a barcode unique to the location of the capture oligonucleotide.

Clause 8. The method of clause 7, wherein each capture oligonucleotide further comprises one or more of: a capture-primer hybridizing region, a capture-detector hybridizing region, a capture-splint hybridizing region, a cleavable linker, a unique molecular index (UMI), a sequencing platform adaptor construct, and a detectable label.

Clause 9. The method of clause 8, wherein the cleavable linker is a photo-cleavable linker.

Clause 10. The method of clause 8, wherein the cleavable linker is chemically cleavable.

Clause 11. The method of any of preceding clauses, wherein the addressable array of capture oligonucleotides comprises unique spots of capture oligonucleotides, wherein each spot has a longest dimension that is 200 μm or less.

Clause 12. The method of any of preceding clauses, wherein the addressable array of capture oligonucleotides has dimensions selected from the group consisting of: 75 mm×25 mm, 75×50 mm, 46×27 mm, 48×28 mm, and 18×18 mm.

Clause 13. The method of clause 11 or 12, wherein the addressable array of capture oligonucleotides comprises unique spots of capture oligonucleotides in a number ranging from 150 to 50,000.

Clause 14. The method of any of clauses 11 to 13, wherein the addressable array of capture oligonucleotides has a density of unique spots of capture oligonucleotides ranging from 2000 to 4 million per square centimeter.

Clause 15. The method of any of preceding clauses, wherein capture oligonucleotides of the oligonucleotide indexed surface range in length from 30 and 100 nucleotides.

Clause 16. The method of any of preceding clauses, wherein the oligonucleotide indexed surface comprises a solid support of a material selected from the group consisting of glass, nitrocellulose, silicon, plastic, and combinations thereof.

Clause 17. The method of clause 16, wherein the oligonucleotide indexed surface comprises a solid support of plastic.

Clause 18. The method of clause 17, wherein the plastic comprises a polymer selected from the group consisting of polystyrene, polycarbonate, polyvinyl chloride, polypropylene, and combinations thereof.

Clause 19. The method of any of preceding clauses, wherein the contacting the biological sample section with the oligonucleotide indexed surface comprises laying the biological sample section onto the oligonucleotide indexed surface.

Clause 20. The method of clause 19, further comprising laying a second oligonucleotide indexed surface onto the biological sample section laid onto the oligonucleotide indexed surface, wherein the second oligonucleotide indexed surface comprises a second addressable array of capture oligonucleotides.

Clause 21. The method of any of preceding clauses, wherein the analyte is a selected from the group consisting of: protein, DNA, RNA, lipid, or carbohydrate.

Clause 22. The method of any of preceding clauses, wherein the analyte-specific binding member is selected from the group consisting of: antibody, oligonucleotide, aptamer, polypeptide, carbohydrate, lipid, or small molecule.

Clause 23. The method of any of preceding clauses, wherein the detector oligonucleotide comprises a barcode unique to the analyte.

Clause 24. The method of clause 23, wherein the detector oligonucleotide further comprises one or more of: a detector-primer hybridizing region, a detector-capture hybridizing region, unique molecular index (UMI), a detector-splint hybridizing region, a cleavable linker, a sequencing platform adaptor, and a detectable label.

Clause 25. The method of clause 24, wherein the cleavable linker is a photo-cleavable linker.

Clause 26. The method of clause 24, wherein the cleavable linker is chemically cleavable.

Clause 27. The method of any of preceding clauses, wherein the detector oligonucleotide has a length ranging from 30 to 100 nucleotides.

Clause 28. The method of any of preceding clauses, wherein probing the oligonucleotide indexed surface contacted biological sample section with the analyte-specific binding member comprises incubating the biological sample section with the analyte-specific binding member for duration and under conditions that allow binding of the analyte-specific binding member to the analyte.

Clause 29. The method of clause 28, further comprising washing the biological sample section to remove any unbound analyte-specific binding member.

Clause 30. The method of any of preceding clauses, wherein linking the detector oligonucleotide to the capture oligonucleotide proximal thereto to produce the linked product nucleic acid comprises ligating the detector oligonucleotide to the capture oligonucleotide to produce a ligated product nucleic acid.

Clause 31. The method of clause 30, wherein ligating the detector oligonucleotide to the capture oligonucleotide proximal thereto to produce the ligated product nucleic acid comprises hybridizing a splint oligonucleotide to: 1) a capture-splint hybridizing region on the capture oligonucleotide via a capture hybridizing region on the splint oligonucleotide that is complementary to capture-splint hybridizing region and 2) a detector-splint hybridizing region on the detector oligonucleotide via a detector hybridizing region on the splint oligonucleotide that is complementary to the detector-splint hybridizing region.

Clause 32. The method of clause 31, further comprising ligating via a ligase the capture-splint hybridizing region of the capture oligonucleotide and the detector-splint hybridizing region of the detector oligonucleotide.

Clause 33. The method of any of clauses 1 to 29, wherein linking the detector oligonucleotide to the capture oligonucleotide proximal thereto to produce the linked product nucleic acid comprises hybridizing the detector oligonucleotide to the capture oligonucleotide to produce a linked product oligonucleotide, the hybridizing performed via a capture-detector hybridizing region on the capture oligonucleotide that hybridizes with a detector-capture hybridizing region on the detector oligonucleotide.

Clause 34. The method of clause 33, wherein the linked oligonucleotide comprises a double stranded region of hybridized portions from the capture and detector oligonucleotides and single stranded overhangs of unhybridized portions of the capture and detector oligonucleotides.

Clause 35. The method of any of clause 33 or 34, further comprising extending one or both of the hybridized capture oligonucleotide and detector oligonucleotide using a polymerase to produce an extension product nucleic acid.

Clause 36. The method of any of clauses 33 to 35, comprising amplifying only one of the capture oligonucleotides and the detector oligonucleotide using a polymerase.

Clause 37. The method of clause 36, wherein the 3′ end of the capture oligonucleotide or the detector oligonucleotide that is not amplified using the polymerase is modified to prevent the polymerase amplification.

Clause 38. The method of clause 37, wherein the modification to prevent the polymerase amplification comprises introducing a 3′ NH₂ group at the end of the oligonucleotide.

Clause 39. The method of any of preceding clauses, wherein the sequencing comprises amplifying the linked product nucleic acid via a polymerase chain reaction using a capture-primer and/or a detector-primer and sequencing the amplified one or more linked oligonucleotides.

Clause 40. The method of clause 39, wherein sequencing comprises amplifying the linked product nucleic acid attached to the oligonucleotide indexed surface.

Clause 41. The method of clause 39, wherein sequencing comprises amplifying the linked product nucleic acid cleaved from the oligonucleotide indexed surface. Clause 42. The method of any of preceding clauses, wherein sequencing comprises a next generation sequencing.

Clause 43. The method of clause 42, wherein the next generation sequencing comprises paired-end sequencing, ion-proton sequencing, pyrosequencing, nanopore sequencing.

Clause 44. The method of any of preceding clauses, further comprising:

probing the oligonucleotide indexed surface contacted biological sample section with one or more additional analyte-specific binding members, wherein each additional analyte-specific binding member specifically binds to an additional analyte, and wherein each of the one or more additional analyte-specific binding members comprises an additional detector oligonucleotide, each additional detector oligonucleotide comprising a barcode unique to the additional analyte.

Clause 45. The method of clause 44, further comprising linking the one or more additional detector oligonucleotides to capture oligonucleotides proximal thereto to produce one or more additional linked product nucleic acids.

Clause 46. The method of clause 44, wherein linking the one or more additional detector oligonucleotides to the capture oligonucleotides proximal thereto to produce the one or more additional linked product nucleic acids comprises ligating the one or more additional detector oligonucleotides to the capture oligonucleotides proximal thereto to produce the one or more additional ligated product nucleic acids.

Clause 47. The method of clause 46, wherein ligating the additional one or more detector oligonucleotides to the capture oligonucleotides proximal thereto to produce the one or more ligated product nucleic acids comprises hybridizing a splint oligonucleotide to: 1) the capture-splint hybridizing region on the capture oligonucleotide via a capture hybridizing region on the splint oligonucleotide that is complementary to capture-splint hybridizing region and 2) the detector-splint hybridizing region on the one or more additional detector oligonucleotides via a detector hybridizing region on the splint oligonucleotide that is complementary to the detector-splint hybridizing region.

Clause 48. The method of clause 47, further comprising ligating via a ligase the capture-splint hybridizing region of the capture oligonucleotide and the detector-splint hybridizing regions of the one or more additional detector oligonucleotides.

Clause 49. The method of any of clauses 1 to 44, wherein linking the one or more additional detector oligonucleotides to the capture oligonucleotides proximal thereto to produce one or more additional linked product nucleic acids comprises hybridizing the one or more additional detector oligonucleotides to the capture oligonucleotide to produce the one or more additional linked product oligonucleotides, the hybridizing performed via a capture-detector hybridizing region on the capture oligonucleotide that hybridizes with a detector-capture hybridizing region on the one or more additional detector oligonucleotides.

Clause 50. The method of clause 49, wherein the one or more additional linked oligonucleotides comprise a double stranded region of hybridized portions from the capture and the one or more additional detector oligonucleotides and single stranded overhangs of unhybridized portions of the capture and detector oligonucleotides.

Clause 51. The method of any of clause 49 or 50, further comprising amplifying one or both of the hybridized capture oligonucleotides and the one or more additional detector oligonucleotides using a polymerase to produce copies of the one or more additional linked product nucleic acids.

Clause 52. The method of any of clauses 49 to 51, comprising amplifying only one of the capture oligonucleotides and the one or more additional detector oligonucleotides using a polymerase.

Clause 53. The method of clause 52, wherein the 3′ end of the capture oligonucleotide or the one or more additional detector oligonucleotides that is not amplified using the polymerase is modified to prevent the polymerase amplification.

Clause 54. The method of clause 53, wherein the modification to prevent the polymerase amplification comprises introducing a 3′ NH₂ group at the end of the oligonucleotide.

Clause 55. The method of clause 45 to 54, further comprising sequencing the one or more additional linked product nucleic acids to assess the biological sample for the one or more additional analytes.

Clause 56. The method of any clauses 44 to 55, wherein each of the one or more additional analytes is a protein, DNA, RNA, lipid, or carbohydrate.

Clause 57. The method of any of clauses 44 to 56, comprising contacting the biological sample section with 10 to 1000 additional analyte-specific binding members.

Clause 58. The method of any of clauses 44 to 57, wherein each of the one or more additional binding members is an antibody, oligonucleotide, aptamer, polypeptide, carbohydrate, lipid, or small molecule.

Clause 59. The method of any of clauses 44 to 58, wherein the biological sample section is contacted simultaneously with the analyte-specific binding member and the one or more additional analyte-specific binding members.

Clause 60. The method of any of clauses 44 to 58, wherein the biological sample section is contacted sequentially with the analyte-specific binding member and the one or more additional analyte-specific binding members.

Clause 61. The method of any of clauses 45 to 60, further comprising amplifying the linked product nucleic acid and the one or more additional linked product nucleic acids via a polymerase chain reaction using a capture-primer and/or a detector-primer and sequencing the amplified linked product nucleic acid and the one or more additional linked product nucleic acids.

Clause 62. The method of clause 61, comprising amplifying the linked product nucleic acid and the one or more additional linked product nucleic acids attached to the oligonucleotide indexed surface.

Clause 63. The method of clause 61, comprising amplifying the linked product nucleic acid and the one or more additional linked product nucleic acids cleaved from the oligonucleotide indexed surface.

Clause 64. The method of any of clauses 61 to 63, wherein sequencing comprises a next generation sequencing.

Clause 65. The method of clause 64, wherein the next generation sequencing comprises paired-end sequencing, ion-proton sequencing, pyrosequencing, or nanopore sequencing.

Clause 66. The method of any of clauses 1 to 43, further comprising determining the location of the analyte in the biological sample section based on the presence in the linked product nucleic acid of the barcodes unique to the capture oligonucleotides and the barcode unique to the detector oligonucleotide.

Clause 67. The method of any of the preceding clauses, further comprising determining the location of the analyte and the one or more additional analytes in the biological sample section based on the presence in the linked product nucleic acid and the one or more additional linked product nucleic acids of the barcodes unique to the capture oligonucleotides and the barcodes unique to the detector oligonucleotide and/or the one or more additional detector oligonucleotides.

Clause 68. A method comprising:
a) determining, according to clause 66, a location of an analyte in a plurality of biological sample sections of a three-dimensional biological sample, and
b) ascertaining the location of the analyte in the three-dimensional biological sample section by stacking the determined location of the analyte in the plurality of biological sample sections of the three-dimensional biological sample.

Clause 69. A method comprising:
a) determining, according to clause 67, a location of an analyte and one or more additional analytes in a plurality of biological sample sections of a three-dimensional biological sample, and
b) ascertaining the location of the analyte and/or one or more additional analytes in the three-dimensional biological sample section by stacking the determined location of the analyte and/or one or more additional analytes in the plurality of biological sample sections of the three-dimensional biological sample.

Clause 70. The method of any of preceding clauses, further comprising evaluating histology, pathology, or morphology of the biological sample section based on the location of the analyte and/or one or more additional analytes in the biological sample.

Clause 71. A kit comprising:
a) an oligonucleotide indexed surface comprising an addressable array of capture oligonucleotides; and
b) an analyte-specific binding member that specifically binds to the analyte, wherein the analyte-specific binding member comprises a detector oligonucleotide.

Clause 72. The kit of clause 71, wherein each capture oligonucleotide in the oligonucleotide indexed surface comprises a barcode unique to the location of the capture oligonucleotide.

Clause 73. The kit of clause 72, wherein each capture oligonucleotide further comprises one or more of: a capture-detector hybridizing region, a capture-primer hybridizing region, a capture-splint hybridizing region, a cleavable linker, a unique molecular index, a sequencing platform adaptor construct, and a detectable label.

Clause 74. The kit of clause 73, wherein the cleavable linker is a photo-cleavable linker.

Clause 75. The kit of clause 74, wherein the cleavable linker is chemically cleavable.

Clause 76. The kit of any of clauses 71 to 75, wherein the addressable array of capture oligonucleotides comprises spots of capture oligonucleotides having a longest dimension of 200 μm or less.

Clause 77. The kit of any of clause 76, wherein the addressable array of capture oligonucleotides comprises a number of unique spots of capture oligonucleotides ranging from 150 to 50,000.

Clause 78. The kit of any of clauses 71 to 77, wherein the addressable array of capture oligonucleotides has the dimension of selected from the group consisting of: 75 mm×25 mm, 75×50 mm, 46×27 mm, 48×28 mm, or 18×18 mm.

Clause 79. The kit of any of clauses 71 to 78, wherein the addressable array of capture oligonucleotides has a density of unique spots of capture oligonucleotides ranging from 2000 to 4 million per square centimeter.

Clause 80. The kit of any of clauses 71 to 79, wherein capture oligonucleotides in the oligonucleotide indexed surface have a length ranging from 30 to 100 nucleotides.

Clause 81. The kit of any of clauses 71 to 80, wherein the oligonucleotide indexed surface comprises a solid support of a material selected from the group consisting of glass, nitrocellulose, silicon, plastic, and combinations thereof.

Clause 82. The kit of any of clauses 71 to 81, wherein the oligonucleotide indexed surface comprises a solid support of plastic.

Clause 83. The kit of any of clause 82, wherein the plastic comprises a polymer selected from the group consisting of polystyrene, polycarbonate, polyvinyl chloride, polypropylene, and combinations thereof.

Clause 84. The kit of any of clauses 71 to 83, wherein the binding member that specifically binds to the analyte is an antibody, oligonucleotide, aptamer, polypeptide, carbohydrate, lipid, or small molecule.

Clause 85. The kit of any of clauses 71 to 84, wherein the detector oligonucleotide comprises a barcode unique to the analyte.

Clause 86. The kit of clause 86, wherein the detector oligonucleotide further comprises one or more of: a detector-capture hybridizing region, a detector-primer hybridizing region, a detector-splint hybridizing region, a cleavable linker, a sequencing platform adaptor construct, a unique molecular index, and a detectable label.

Clause 87. The kit of clause 86, wherein the cleavable linker is a photo-cleavable linker.

Clause 88. The kit of clause 86, wherein the cleavable linker is chemically cleavable.

Clause 89. The kit of any of clauses 71 to 88, wherein the detector oligonucleotide has a length ranging from 30 to 100 nucleotides.

Clause 90. The kit of any of clauses 71 to 89, further comprising a splint oligonucleotide comprising: 1) a capture hybridizing region complementary to capture-splint hybridizing region and 2) a detector region complementary to the detector-splint hybridizing region.

Clause 91. The kit of any of clauses 71 to 90, further comprising a ligase or a polymerase.

Clause 92. The kit of any of clauses 71 to 91, further comprising at least one of a capture-primer and a detector-primer.

Clause 93. The kit of any of clauses 71 to 92, further comprising: one or more additional analyte-specific binding members, wherein each additional analyte-specific binding member specifically binds to an additional analyte, and wherein each of the one or more additional analyte-specific binding members comprises an additional detector oligonucleotide.

Clause 94. The kit of clause 93, wherein each additional detector oligonucleotide comprises a barcode unique to the additional analyte.

Clause 95. The kit of clause 94, wherein each additional detector oligonucleotide further comprises one or more of: a detector-capture hybridizing region, a detector-primer hybridizing region, a detector-splint hybridizing region, a cleavable linker, a unique molecular index, a sequencing platform adaptor construct, and a detectable label.

Clause 96. The kit of any of clauses 93 to 95, wherein each of the one or more additional binding members is an antibody, oligonucleotide, aptamer, polypeptide, carbohydrate, lipid, or small molecule.

Clause 97. An oligonucleotide indexed surface comprising an addressable array of capture oligonucleotides.

Clause 98. The oligonucleotide indexed surface of clause 97, wherein each capture oligonucleotide in the oligonucleotide indexed surface comprises a barcode unique to the location of the capture oligonucleotide.

Clause 99. The oligonucleotide indexed surface of clause 98, wherein each capture oligonucleotide further comprises one or more of: a capture-detector hybridizing region, a capture-primer hybridizing region, a capture-splint hybridizing region, a cleavable linker, a unique molecular index (UMI), a sequencing platform adaptor construct, and a detectable label.

Clause 100. The oligonucleotide indexed surface of clause 99, wherein the cleavable linker is a photo-cleavable linker.

Clause 101. The oligonucleotide indexed surface of clause 99, wherein the cleavable linker is chemically cleavable.

Clause 102. The oligonucleotide indexed surface of any of clauses 97 to 101, wherein the addressable array of capture oligonucleotides comprises spots of capture oligonucleotides having a longest dimension of 200 μm or less.

Clause 103. The oligonucleotide indexed surface of any of clause 102, wherein the addressable array of capture oligonucleotides comprises a number of unique spots of capture oligonucleotides ranging from 150 to 50,000.

Clause 104. The oligonucleotide indexed surface of any of clauses 97 to 103, wherein the addressable array of capture oligonucleotides has the dimension selected from the group consisting of 75 mm×25 mm, 75×50 mm, 46×27 mm, 48×28 mm, and 18×18 mm.

Clause 105. The oligonucleotide indexed surface of any of clauses 102 to 104, wherein the addressable array of capture oligonucleotides has a density of unique spots of capture oligonucleotides ranging from 2000 to 4 million per square centimeter.

Clause 106. The oligonucleotide indexed surface of any of clauses 97 to 105, wherein capture oligonucleotides in the oligonucleotide indexed surface have a length ranging from 30 to 100 nucleotides.

Clause 107. The oligonucleotide indexed surface of any of clauses 97 to 106, wherein the oligonucleotide indexed surface comprises a solid support of a material selected from the group consisting of glass, nitrocellulose, silicon, plastic, and combinations thereof.

Clause 108. The oligonucleotide indexed surface of any of clauses 97 to 107, wherein the oligonucleotide indexed surface comprises a solid support of plastic.

Clause 109. The oligonucleotide indexed surface of clause 108, wherein the plastic comprises a polymer selected from the group consisting of polystyrene, polycarbonate, polyvinyl chloride, polypropylene, and combinations thereof.

Clause 110. An analyte-specific binding member that specifically binds to the analyte, wherein the analyte-specific binding member comprises an analyte-specific binding domain and a detector oligonucleotide.

Clause 111. The analyte-specific binding member of clause 110, wherein the binding member is an antibody, oligonucleotide, aptamer, polypeptide, carbohydrate, lipid, or small molecule.

Clause 112. The analyte-specific binding member of clause 110 or 111, wherein the detector oligonucleotide comprises a barcode unique to the analyte.

Clause 113. The analyte-specific binding member of clause 112, wherein the detector oligonucleotide further comprises one or more of: a detector-primer hybridizing region, a detector-splint hybridizing region, detector-capture hybridizing region, a cleavable linker, a unique molecular index (UMI), a sequencing platform adaptor construct, and a detectable label.

Clause 114. The analyte-specific binding member of clause 113, wherein the cleavable linker is a photo-cleavable linker.

Clause 115. The analyte-specific binding member of clause 113, wherein the cleavable linker is chemically cleavable.

Clause 116. The analyte-specific binding member of any of clauses 110 to 115, wherein the detector oligonucleotide has a length ranging from 30 to 100 nucleotides.

Clause 117. A plurality of analyte-specific binding members, wherein each analyte-specific binding member binds to a unique analyte, wherein each analyte-specific binding member comprises an analyte-specific binding domain and a detector oligonucleotide.

Clause 118. The plurality of analyte-specific binding members according to clause 117, wherein the detector oligonucleotide comprises a barcode unique to the corresponding analyte.

Clause 119. A system comprising, a processing module configured to receive the following data:

i) an image of a biological sample section,

ii) sequences of capture oligonucleotides in an addressable array of capture oligonucleotides, wherein each capture oligonucleotide comprises a barcode unique to the location of the capture oligonucleotide on the array, and

iii) sequences of linked product nucleic acids obtained by processing the biological sample section according to any of clauses 1 to 43,

wherein the processing module is configured to process the received data to determine the location of the analyte in the biological sample section.

Clause 120. A system comprising,

a processing module configured to receive the following data:
i) a plurality of images of a plurality of sections of a three-dimensional biological sample,
ii) sequences of capture oligonucleotides in a plurality of addressable arrays of capture oligonucleotides, wherein each capture oligonucleotide comprises a barcode unique to the location of the capture oligonucleotide on the plurality of arrays, and
iii) sequences of linked product nucleic acid and/or additional linked product nucleic acids obtained by processing the plurality of sections of the three-dimensional biological sample according to any of clauses 1 to 67,

wherein the processing module is configured to process the received data to determine the location of the analyte and/or the one or more additional analytes in the three-dimensional biological sample by stacking the determined location of the analyte and/or the one or more additional analytes in the plurality of sections of the three-dimensional biological sample.

Clause 121. The system of clause 119 or 120, wherein the signal processing module is further configured to evaluate histology, pathology, or morphology of the biological sample based on the location of the one or more analytes in the biological sample.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

1. A method of assessing a biological sample section for an analyte, the method comprising:
a) contacting the biological sample section with an oligonucleotide indexed surface comprising an addressable array of capture oligonucleotides;
b) probing the oligonucleotide indexed surface contacted with the biological sample section with an analyte-specific binding member that specifically binds to the analyte, wherein the analyte-specific binding member comprises a detector oligonucleotide;
c) linking the detector oligonucleotide to a capture oligonucleotide proximal thereto to produce a linked product nucleic acid, which linked product nucleic acid may be: i) a ligated product nucleic acid made up ligated detector and capture oligonucleotides, or ii) an extension product nucleic acid produced by template mediated extension of hybridized detector and barcode capture oligonucleotides; and
d) sequencing the linked product nucleic acid to assess the biological sample for the analyte.

2. The method of claim 1, wherein the biological sample section is a paraffin-embedded section or a frozen section.

3. The method of claim 1, wherein each capture oligonucleotide in the oligonucleotide indexed surface comprises a barcode unique to the location of the capture oligonucleotide.

4. The method of claim 3, wherein each capture oligonucleotide further comprises one or more of: a capture-primer hybridizing region, a unique molecular index (UMI), a capture-detector hybridizing region, a capture-splint hybridizing region, a cleavable linker, a sequencing platform adapter construct and a detectable label.

5. The method of claim 1, wherein the addressable array of capture oligonucleotides comprises unique spots of capture oligonucleotides, wherein each spot has a longest dimension that is 200 μm or less.

6. The method of claim 1, wherein the analyte is a selected from the group consisting of: protein, DNA, RNA, lipid, or carbohydrate and the analyte-specific binding member is selected from the group consisting of: antibody, oligonucleotide, aptamer, polypeptide, carbohydrate, lipid, or small molecule.

7. The method of claim 1, wherein the detector oligonucleotide comprises a barcode unique to the analyte.

8. The method of claim 7, wherein the detector oligonucleotide further comprises one or more of: a detector-primer hybridizing region, a detector-capture hybridizing region, a unique molecular index (UMI), a detector-splint hybridizing region, a cleavable linker, a sequencing platform adaptor construct, and a detectable label.

9. The method of claim 1, wherein linking the detector oligonucleotide to the capture oligonucleotide proximal thereto to produce the linked product nucleic acid comprises ligating the detector oligonucleotide to the capture oligonucleotide to produce a ligated product nucleic acid.

10. The method of claim 9, wherein ligating the detector oligonucleotide to the capture oligonucleotide proximal thereto to produce the ligated product nucleic acid comprises: (a) hybridizing a splint oligonucleotide to: i) a capture-splint hybridizing region on the capture oligonucleotide via a capture hybridizing region on the splint oligonucleotide that is complementary to capture-splint hybridizing region and ii) a detector-splint hybridizing region on the detector oligonucleotide via a detector hybridizing region on the splint oligonucleotide that is complementary to the detector-splint hybridizing region; and (b) ligating via a ligase the capture-splint hybridizing region of the capture oligonucleotide and the detector-splint hybridizing region of the detector oligonucleotide.

11. The method of claim 1, wherein the linked product nucleic acid comprises an extension product nucleic acid produced by template mediated extension of hybridized detector and capture oligonucleotides.

12. The method of claim 1, wherein the sequencing comprises amplifying the linked product nucleic acid via a polymerase chain reaction using a capture-primer and/or a detector-primer and sequencing the amplified one or more linked oligonucleotides.

13. The method of claim 1, wherein sequencing comprises a next generation sequencing.

14. A kit comprising:
a) an oligonucleotide indexed surface comprising an addressable array of capture oligonucleotides; and
b) an analyte-specific binding member that specifically binds to the analyte, wherein the analyte-specific binding member comprises a detector oligonucleotide.

15. The kit of claim 14, further comprising a ligase or a polymerase.

16. The kit of claim 14, wherein each capture oligonucleotide in the oligonucleotide indexed surface comprises a barcode unique to the location of the capture oligonucleotide.

17. The kit of claim 14, wherein each capture oligonucleotide further comprises one or more of: a capture-detector hybridizing region, a capture-primer hybridizing region, a capture-splint hybridizing region, a cleavable linker, a unique molecular index, a sequencing platform adaptor construct, and a detectable label.

18. The kit of claim 14, wherein the detector oligonucleotide comprises a barcode unique to the analyte.

19. The kit of claim 14, wherein the detector oligonucleotide further comprises one or more of: a detector-capture hybridizing region, a detector-primer hybridizing region, a detector-splint hybridizing region, a cleavable linker, a sequencing platform adaptor construct, a unique molecular index, and a detectable label.

20. The kit of claim 14, further comprising a splint oligonucleotide comprising: 1) a capture hybridizing region complementary to capture-splint hybridizing region and 2) a detector region complementary to the detector-splint hybridizing region.


SEQUENCE LISTING (14 sequences)

SEQ ID NO: 1 - DNA, 20 bp, Artificial sequence
  synthetic sequence
aatgatacggcgaccaccga

SEQ ID NO: 2 - DNA, 24 bp, Artificial sequence
  synthetic sequence
caagcagaagacggcatacgagat

SEQ ID NO: 3 - DNA, 33 bp, Artificial sequence
  synthetic sequence
acactctttccctacacgacgctcttccgatct

SEQ ID NO: 4 - DNA, 34 bp, Artificial sequence
  synthetic sequence
gtgactggagttcagacgtgtgctcttccgatct

SEQ ID NO: 5 - DNA, 30 bp, Artificial sequence
  synthetic sequence
ccatctcatccctgcgtgtctccgactcag

SEQ ID NO: 6 - DNA, 23 bp, Artificial sequence
  synthetic sequence
cctctctatgggcagtcggtgat

SEQ ID NO: 7 - DNA, 74 bp, Artificial sequence
  synthetic sequence
  misc_feature (14)..(29): n is a, c, g, or t
  misc_feature (74)..(74): The nucleotide is attached to 3ThioMC3-D.
tcgtgtctaatatnnnnnnnnnnnnnnnncctccgaaggaagatcggaagagcgtcgtgtagggtttttttttt

SEQ ID NO: 8 - DNA, 86 bp, Artificial sequence
  synthetic sequence
  misc_feature (1)..(1): The nucleotide is attached to 5BiotinTEG.
tttttttttttttttttcagacgtgtgctcttccgatctaacttatacgggacgaaccgctttgcctgactgatcgctaaatcgtg

SEQ ID NO: 9 - DNA, 26 bp, Artificial sequence
  synthetic sequence
  misc_feature (26)..(26): The nucleotide is attached to 3AmMO.
tacttagacacgacacgatttagttt

SEQ ID NO: 10 - DNA, 21 bp, Artificial sequence
  synthetic sequence
ttcagacgtgtgctcttccga

SEQ ID NO: 11 - DNA, 18 bp, Artificial sequence
  synthetic sequence
accctacacgacgctctt

SEQ ID NO: 12 - DNA, 136 bp, Artificial sequence
  synthetic sequence
  misc_feature (85)..(100): n is a, c, g, or t
ttcagacgtgtgctcttccgatctaacttatacgggacgaaccgctttgcctgactgatcgctaaatcgtgtcgtgtctaatatnnnnnnnnnnnnnnnncctccgaaggaagatcggaagagcgtcgtgtagggt

SEQ ID NO: 13 - DNA, 19 bp, Artificial sequence
  synthetic sequence
atgatgatagggatctccg

SEQ ID NO: 14 - DNA, 63 bp, Artificial sequence
  synthetic sequence
  misc_feature (30)..(40): n is a, c, g, or t
tttttttttttttttttcagacgtgtgctnnnnnnnnnnngcctgactgatcgctaaatcgtg